(19) United States
(12) Patent Application Publication
(10) Pub. No.: US 2004/0067499 A1
(43) Pub. Date: Apr. 8, 2004

(54) Novel g protein-coupled receptor

(76) Inventors: Tatsuya Haga, Zushi-shi, Kanagawa (JP); Shigeki Takeda, Yokohama-shi (JP); Shigeki Takeda, Kokubunj-shi, Tokyo (JP)

Correspondence Address: VENABLE, BAETJER, HOWARD AND CIVILETTI, LLP, P.O. BOX 34385, WASHINGTON, DC, 20043-9998, US

(21) Appl. No.: 10/343,650

(22) PCT Filed: Jul. 30, 2001
(86) PCT No.: PCT/IB01/01446

(30) Foreign Application Priority Data
Aug. 4, 2000 (JP) 2000-237818

(51) Int. Cl. C12Q 1/68; G01N 33/53; G06F 19/00; G01N 33/48; G01N 33/50

(52) U.S. Cl. 435/6; 435/7.1; 702/19

(57) ABSTRACT

The present invention provides a method by which, novel G protein-coupled receptor (GPCR) genes or GPCR proteins which could discover the biocommunication system or identify novel target proteins for medicine can be searched for cyclopaedically on a data base. The method comprises the steps of: extracting open reading frames (ORFs) comprising 250 to 1000 amino acid residues from the human genome data; eliminating ORFs derived from repeated sequences of DNA, ORFs with substantial undetermined amino acids, and ORFs in which any single amino acid residue comprising more than 20% of the whole sequence; extracting ORFs having 6 to 8 transmembrane segments using SOSUI; and searching the obtained ORFs for genes homologous to known GPCR genes, preferably, genes wherein their highest homologous genes in homology search are GPCR genes or GPCR associated genes.

### TECHNICAL FIELD

The present invention relates to a method by which a novel G protein-coupled receptor gene and a novel G protein-coupled receptor protein can be searched for cyclopaedically on a data base, a novel G protein-coupled receptor gene and a novel G protein-coupled receptor obtained by said method, and a screening method of an endogenous and an exogenous ligand such as an agonist or an antagonist of said gene and protein.

### BACKGROUND ART

G protein-coupled receptors (GPCRs), which exist on the cell membrane, are proteins receiving various extracellular information. GPCRs comprise superfamilies having a common structure of seven transmembrane segments. It is known that each GPCR works as a sensor to physical senses such as light, odor, and taste, or, by binding to extracellular ligands such as hormones, neurotransmitters, biologically active substances, and local mediater and the like, changes conformation of these receptors, activates a G protein (GTP-binding protein) such as Gi, Gt, Gs, Go, Gq, and G12, and transmits signals to the intercellular space. Further, extracellular ligands, which control complex internal functions of cells and organs of various living organisms, are utilized as pharmaceuticals to control vital functions. It is considered that 30-50% of the presently used clinical medicines are ligands targeting GPCRS. Recently, the development of the gene cloning technology has made it possible to find many genes of “Orphan GPCRs ” whose ligands have not yet been identified and the search for novel GPCRs and the elucidation of the function are required. Cloning of these novel GPCR genes are considered to be useful to search for ligands specific to GPCRs, such as agonists and antagonists and the like. However, not all of GPCRs have been clarified.

On the other hand, SOSUI (http://sosui.proteome.bio.tuat.ac.jp/sosuiframeO.html) is a program developed to predict transmembrane segments from the primary structure of proteins using a hydrophobic parameter and the like of each amino acid. SOSUI has already been applied to predict the structure of many known and unknown proteins, and also used to identify a translation segment (ORF: open reading frame) which encodes a membrane-bound protein on the genome gene sequence.

GPCRs and their endogenous ligands are expected to be applicable to the research of medicines acting on them, and to the novel therapies, such as the application to gene therapies using said gene and its mutant. Moreover, analysis of novel GPCR genes is expected to lead to discover a new biocommunication system, as well as to identify a novel target protein for medicine. The object of this invention is to provide a searching method by which novel GPCR genes or GPCR proteins, which enable to discover a biocommunication system or identify a novel target protein for medicine, can be searched for cyclopaedically on a data base.

### DISCLOSURE OF THE INVENTION

In order to attain the above-mentioned object, after having attempted several methods to search for GPCR genes cyclopaedically on a database, the inventors of the present invention extracted translation segments (ORFs: Open Reading Frames) which could be candidates for GPCR genes from the human genome data, using the structural feature that GPCRs have seven transmembrane segments, and the fact that many known GPCR genes have no introns in their translation segments, then analyzed the extracts by SOSUI. Further, undetermined bases in the genome sequence were made to be translated into most possible amino acids that could become transmembrane segments, when translating into the amino acids. Analysis by SOSUI has a possibility of interpreting a signal peptide of GPCRs as a transmembrane segment, or not recognizing seventh transmembrane segment as a transmembrane segment due to its tendency of low hydrophobicity in the structure of GPCRs. Taking these into consideration, ORFs which were predicted to have 6 to 8 transmembrane segments were finally selected as candidates for GPCRs, and it is found that novel GPCR genes can be identified by the homology search of these candidate genes and known GPCR genes. The present invention has thus been completed.

The present invention relates to a searching method of a G protein-coupled receptor gene and/or a G protein-coupled receptor protein characterized in extracting an open reading frame comprising 200 to 1500 amino acid residues and having 6 to 8 transmembrane segments from the genome data derived from a human, and in searching for a gene homologous to a known G protein-coupled receptor gene from the obtained open reading frame (claim 1), the searching method of a G protein-coupled receptor gene and/or a G protein-coupled receptor protein according to claim 1, wherein an open reading frame derived from a repeated sequence of DNA, an open reading frame with substantial undetermined amino acids, and an open reading frame in which any single amino acid residue comprising more than 20% of the whole sequence are eliminated, when extracting the open reading frame (claim 2), the searching method of a G protein-coupled receptor gene and/or a G protein-coupled receptor protein according to claim 1 or 2, wherein a gene homologous to a known G protein-coupled receptor gene is a G protein-coupled receptor gene or a G protein-coupled receptor associated gene (claim 3), the searching method of a G protein-coupled receptor gene and/or a G protein-coupled receptor protein according to any one of claims 1 to 3, wherein a G protein-coupled receptor contains an endogenous ligand (claim 4), the searching method of a G protein-coupled receptor gene and/or a G protein-coupled receptor protein according to claim 4, wherein a G protein-coupled receptor containing an endogenous ligand is a G protein-coupled receptor other than an odorant receptor or a taste receptor (claim 5), the searching method of a G protein-coupled receptor gene and/or a G protein-coupled receptor protein according to claim 4, wherein a G protein-coupled receptor containing an endogenous ligand is a G protein-coupled receptor of an odorant receptor (claim 6), and the searching method of a G protein-coupled receptor gene and/or a G protein-coupled receptor protein according to claim 4, wherein a G protein-coupled receptor containing an endogenous ligand is a G protein-coupled receptor of a taste receptor (claim 7).

The present invention also relates to a G protein-coupled receptor gene obtainable by the searching method of a G protein-coupled receptor gene and/or a G protein-coupled receptor protein according to any one of claims 1 to 7 (claim 8), a gene which encodes a G protein-coupled receptor protein (a) or (b) described below; (a) a G protein-coupled receptor protein comprising an amino acid sequence represented by Seq. ID No.2n (n=any one of integral numbers 1 to 51), (b) a G protein-coupled receptor protein comprising an amino acid sequence where one or a few amino acids are deficient, substituted or added in an amino acid sequence represented by Seq. ID No.2n (n=any one of integral numbers 1 to 51) (claim 9), DNA which encodes a G protein-coupled receptor protein comprising DNA which contains a base sequence represented by Seq. ID No. 2n-1 (n=any one of integral numbers 1 to 51) or its complementary sequence and a part or the whole of these sequences (claim 10), DNA which hybridizes with DNA comprising the gene according to claim 10 under a stringent condition, and encodes a G protein-coupled receptor protein (claim 11), a gene which encodes a G protein-coupled receptor protein (a) or (b) described below; (a) a G protein-coupled receptor protein comprising an amino acid sequence represented by Seq. ID No. 2n (n=any one of integral numbers 52 to 332), (b) a G protein-coupled receptor protein comprising an amino acid sequence where one or a few amino acids are deficient, substituted or added in an amino acid sequence represented by Seq. ID No. 2n (n=any one of integral numbers 52 to 332) (claim 12), DNA which encodes a G protein-coupled receptor protein comprising DNA which contains a base sequence represented by Seq. ID No. 2n-1 (n=any one of integral numbers 52 to 332) or its complementary sequence and a part or the whole of these sequences (claim 13), DNA which hybridizes with DNA comprising the gene according to claim 13 under a stringent condition, and encodes a G protein-coupled receptor protein (claim 14), a gene which encodes a G protein-coupled receptor proteins (a) or (b) described below; (a) a G protein-coupled receptor protein comprising an amino acid sequence represented by Seq. ID No. 2n (n=any one of integral numbers 333 to 347), (b) a G protein-coupled receptor protein comprising an amino acid sequence where one or a few amino acids are deficient, substituted or added in an amino acid sequence represented by Seq. ID No. 2n (n=any one of integral numbers 333 to 347) (claim 15), DNA which encodes a G protein-coupled receptor protein comprising DNA which contains a base sequence represented by Seq. ID No. 2n-1 (n=any one of integral numbers 333 to 347) or its complementary sequence and a part or the whole of these sequences (claim 16), and DNA which hybridizes with DNA comprising the gene according to claim 16 under a stringent condition, and encodes a G protein-coupled receptor protein (claim 17).

The present invention also relates to a G protein-coupled receptor protein obtainable by the searching method of a G protein-coupled receptor gene and/or a G protein-coupled receptor protein according to any one of claims 1 to 7 (claim 18), a G protein-coupled receptor protein comprising an amino acid sequence represented by Seq. ID No. 2n (n=any one of integral numbers 1 to 51) (claim 19), a G protein-coupled receptor protein comprising an amino acid sequence where one or a few amino acids are deficient, substituted or added in an amino acid sequence represented by Seq. ID No. 2n (n=any one of integral numbers 1 to 51) (claim 20), a G protein-coupled receptor protein comprising an amino acid sequence represented by Seq. ID No. 2n (n=any one of integral numbers 52 to 332) (claim 21), a G protein-coupled receptor protein comprising an amino acid sequence where one or a few amino acids are deficient, substituted or added in an amino acid sequence represented by Seq. ID No. 2n (n=any one of integral numbers 52 to 332) (claim 22), a G protein-coupled receptor protein comprising an amino acid sequence represented by Seq. ID No. 2n (n=any one of integral numbers 333 to 347) (claim 23), a G protein-coupled receptor protein comprising an amino acid sequence where one or a few amino acids are deficient, substituted or added in an amino acid sequence represented by Seq. ID No. 2n (n=any one of integral numbers 333 to 347) (claim 24), a partial peptide of a G protein-coupled receptor protein obtainable by the searching method of a G protein-coupled receptor gene and/or a G protein-coupled receptor protein according to any one of claims 1 to 7 (claim 25), and the partial peptide according to claim 25, wherein a G protein-coupled receptor protein is the G protein-coupled receptor protein according to any one of claims 19 to 22 (claim 26).

The present invention also relates to a fusion protein or a fusion peptide constructed by binding the G protein-coupled receptor protein according to claim 18 or the partial peptide of the G protein-coupled receptor protein according to claim 25 to a marker protein and/or a peptide tag (claim 27), the fusion protein according to claim 27, wherein the G protein-coupled receptor protein is the G protein-coupled receptor protein according to any one of claims 19 to 24 (claim 28), an antibody which specifically binds to the G protein-coupled receptor protein according to claim 18 (claim 29), the antibody according to claim 29, wherein the G protein-coupled receptor protein is the G protein-coupled receptor protein according to any one of claims 19 to 24 (claim 30), a host cell containing an expression system which can express the G protein-coupled receptor protein according to claim 18 (claim 31), the host cell according to claim 31, wherein the G protein-coupled receptor protein is the G protein-coupled receptor protein according to any one of claims 19 to 24 (claim 32), a non-human animal wherein its function of a gene which encodes the G protein-coupled receptor protein according to claim 18 is deficient or said protein overexpresses on its chromosome (claim 33), the non-human animal according to claim 33, wherein the G protein-coupled receptor protein is the G protein-coupled receptor protein according to any one of claims 19 to 24 (claim 34), and the non-human animal according to claim 33 or 34, wherein the non-human animal is a mouse (claim 35).

The present invention also relates to a screening method of a promoter or a suppressor of a G protein-coupled receptor function, or of a G protein-coupled receptor expression characterized in using the G protein-coupled receptor protein according to any one of claims 18 to 24, the partial peptide according to claim 25 or 26, or a cell membrane which expresses said protein or the partial peptide, and a test substance (claim 36), a screening method of a promoter or a suppressor of a G protein-coupled receptor function, or of a G protein-coupled receptor expression characterized in using the G protein-coupled receptor protein according to any one of claims 18 to 24, the partial peptide according to claim 25 or 26, or a cell membrane which expresses said protein or the partial peptide, a G protein or a partial peptide of a G protein, and a test substance (claim 37), a screening method of a promoter or a suppressor of a G protein-coupled receptor function, or of a G protein-coupled receptor expression characterized in using a cell expressing the G protein-coupled receptor protein according to any one of claims 18 to 24 or the partial peptide according to claim 25 or 26, and a test substance (claim 38), the screening method of a promoter or a suppressor of a G protein-coupled receptor function, or of a G protein-coupled receptor expression according to any one of claims 36 to 38, wherein a cell which expresses the G protein-coupled receptor protein according to any one of claims 18 to 24 or the partial peptide according to claim 25 or 26 is the host cell according to claim 31 or 32 (claim 39), and a screening method of a promoter or a suppressor of a G protein-coupled receptor function, or of a G protein-coupled receptor expression characterized in using the non-human animal according to any one of claims 33 to 35, and a test substance (claim 40).

The present invention also relates to a promoter or a suppressor of a G protein-coupled receptor function, or of a G protein-coupled receptor expression obtainable by the screening method of a promoter or a suppressor of a G protein-coupled receptor function, or of a G protein-coupled receptor expression according to any one of claims 36 to 40 (claim 41), the promoter or the suppressor of a G protein-coupled receptor function, or of a G protein-coupled receptor expression according to claim 41, wherein the promoter or the suppressor of a G protein-coupled receptor function, or of a G protein-coupled receptor expression is a ligand for a G protein-coupled receptor (claim 42), a medical constituent characterized in being used for a medical treatment for a patient who needs elevation of the function or enhancement of the expression of a G protein-coupled receptor, and containing the protein according to any one of claims 18 to 24, the partial peptide according to claim 25 or 26, or the promoter of a G protein-coupled receptor function or expression according to claim 41 or 42 as an active component (claim 43), and a medical constituent characterized in being used for a medical treatment for a patient who needs suppression of the function or the expression of a G protein-coupled receptor, and containing the protein according to any one of claims 18 to 24, the partial peptide according to claim 25 or 26, or the suppressor of a G protein-coupled receptor function or expression according to claim 41 or 42 as an active component (claim 44).

The present invention also relates to a diagnostic method for diseases relating to the function or the expression of a G protein-coupled receptor characterized in comparing a DNA sequence encoding a G protein-coupled receptor protein in a sample to a DNA sequence encoding the protein according to any one of claims 18 to 24 (claim 45), a diagnostic probe for diseases relating to the function or the expression of a G protein-coupled receptor comprising the whole or a part of an anti sense strand of DNA or RNA encoding the protein according to any one of claims 18 to 24 (claim 46), and a diagnostic drug for diseases relating to the function or the expression of a G protein-coupled receptor characterized in containing the diagnostic probe according to claim 46 and/or the antibody according to claim 29 or 30 (claim 47).

### BEST MODE OF CARRYING OUT THE INVENTION

The searching method of GPCR genes and/or GPCR proteins of the present invention is not limited in particular, as far as it comprises a searching method characterized in extracting open reading frames comprising 200 to 1500 amino acid residues and having 6 to 8 transmembrane segments from the genome data derived from a human, and in searching the obtained open reading frames for genes homologous to known GPCR genes, preferably genes which are GPCR genes or GPCR associated genes, and more preferably genes which are GPCR genes containing endogenous ligands. It is preferable, however, to eliminate open reading frames derived from repeated sequences of DNA, open reading frames with substantial undetermined amino acids, or open reading frames comprising more than 20% of identical amino acid, when extracting open reading frames. In order to extract open reading frames with 6 to 8 transmembrane segments, the above-mentioned program, for example, such as SOSUI, which was developed to predict transmembrane segments of amino acids from the primary structure of proteins using a hydrophobic parameter and the like of each amino acid, can be used. Additionally, in order to search for homologous genes, the known homology search system, such as BLAST, can be used.

As GPCR genes of the present invention, any GPCR genes obtained from the above-mentioned homology search can be used and the examples are genes which encode GPCR proteins other than odorant receptors and taste receptors comprising an amino acid sequence represented by Seq. ID No.2n (n=any one of integral numbers 1 to 51), genes which encode GPCR proteins of odorant receptors comprising an amino acid sequence represented by Seq. ID No. 2n (n=any one of integral numbers 52 to 332), genes which encode GPCR proteins of taste receptors comprising an amino acid sequence represented by Seq. ID No. 2n (n=any one of integral numbers 333 to 347), and genes which encode GPCR proteins comprising an amino acid sequence where one or a few amino acids are deficient, substituted or added in an amino acid sequence represented by Seq. ID No.2n (n=any one of integral numbers 1 to 347). Said GPCR genes can be prepared based on their DNA sequence data. For example, GPCR genes derived from a human can be prepared from a human gene library or a human cDNA library and the like by publicly known methods.

As DNA which encodes GPCR proteins of the present invention, the examples are DNA which contains a base sequence represented by Seq. ID No. 2n-1 (n=any one of integral numbers 1 to 347) or its complementary sequence and a part or the whole of these sequences, and DNA which hybridizes with various DNA libraries under a stringent condition by using said DNA as a probe, and encodes GPCR proteins. As the hybridization condition to obtain said DNA, for instance, a hybridization at 42.degree. C., and a rinse with a buffer containing 1×SSC and 0.1% SDS at 42. degree. C. are exemplified, and more preferably, a hybridization at 65.degree. C., and a rinse with a buffer containing 1×SSC and 0.1% SDS at 65.degree. C. are exemplified. In addition, there are various factors which influence the stringency of hybridization besides the above-mentioned temperature condition, and one skilled in the art can construct the same level of stringency as the above exemplified hybridization stringency, by combining various factors accordingly.

As GPCR proteins of the present invention, any GPCR proteins obtained by the above-mentioned screening method can be used, and the specific examples are GPCR proteins other than odorant receptors and taste receptors comprising an amino acid sequence represented by Seq. ID No. 2n (n=any one of integral numbers 1 to 51), GPCR proteins of odorant receptors comprising an amino acid sequence represented by Seq. ID No. 2n (n=any one of integral numbers 52 to 332), GPCR proteins of taste receptors comprising an amino acid sequence represented by Seq. ID No. 2n (n=any one of integral numbers 333 to 347), GPC.R proteins comprising an amino acid sequence where one or a few amino acids are deficient, substituted or added in an amino acid sequence represented by Seq. ID No.2n (n=any one of integral numbers 1 to 347), and recombinant proteins of said proteins. Further, partial peptides of the above-mentioned GPCR proteins, which are objects of the present invention, are not limited in particular, as far as they comprise a part of the above-mentioned GPCR proteins, and contain an amino acid sequence which recognizes or specifically binds to various G proteins. The above-mentioned objects of the present invention, that is, GPCR proteins, partial peptides of GPCR proteins, and recombinant proteins and peptides to which the antibody, which specifically binds to GPCR proteins and partial peptides of GPCR proteins, specifically binds, may be generically called hereafter as “the protein and the peptide of the present invention”. The protein and the peptide of the present invention can be prepared by publicly known methods based on their DNA sequence data, and the origin is not limited in particular.

As fusion proteins and fusion peptides of the present invention, any fusion proteins and fusion peptides constructed by binding the protein and the peptide of the present invention to a marker protein and/or a peptide tag can be used. As marker proteins, any conventional marker proteins can be used and the specific examples are alkaline phosphatase, Fc region of an antibody, HRP, and GFP. Conventionally known peptide tags, such as Myc tag, His tag, FLAG tag, GST tag, are exemplified as specific examples of peptide tags of the present invention. Said fusion proteins can be constructed by usual methods, and are useful for the purification of GPCR proteins utilizing the affinity between Ni-NTA and His tag, the detection of GPCR proteins, the quantitation of the antibody to GPCR proteins, and as an investigational reagent and the like in the field concerned.

As the antibody that specifically binds to the protein and the peptide of the present invention, an immunospecific antibody such as a monoclonal antibody, a polyclonal antibody, a chimeric antibody, a single stranded antibody, a humanized antibody and the like are concretely exemplified. Although these antibodies can be constructed by usual methods using a protein such as the above-mentioned GPCR proteins or a part of the protein, or the like as an antigen, a monoclonal antibody is more preferable among them because of its specificity. Said antibody which specifically binds to GPCR proteins, such as a monoclonal antibody or the like, are useful, for instance, for the diagnosis of diseases derived from the mutation or the defect of GPCR proteins, and for elucidation of molecular mechanism of GPCR proteins.

The antibody of the present invention can be developed by administering fragments containing the protein and the peptide of the present invention or their epitopes, or cells that express said proteins or peptides on the surface of the membrane, to animals (preferably excluding human) with usual protocol. For instance, the monoclonal antibody can be prepared by an arbitrary method which brings antibodies developed by cultured materials of continuous cell line, such as hybridoma method (Nature 256, 495-497, 1975), trioma method, human B-cell hybridoma method (Immunology Today 4, 72, 1983), and EBV-hybridoma method (MONOCLONAL ANTIBODIES AND CANCER THERAPY, pp. 77-96, Alan R. Liss, Inc., 1985). Taking GPCR proteins derived from a mouse as examples of the protein and the peptide of the present invention, the preparing method of the monoclonal antibody that specifically binds to GPCR proteins derived from a mouse, in other word, anti-m GPCR (mouse GPCR) monoclonal antibody is now explained.

The above-mentioned anti-mGPCR monoclonal antibody can be produced by culturing the hybridoma developing anti-mGPCR monoclonal antibody by usual methods in vivo or in vitro. In the in vivo system, for instance, said antibody can be obtained by intraperitoneal culturing in a rodent, preferably in a mouse or a rat, and in the in vitro system, by culturing in an animal cell culture medium. The cell culture media such as RPMI1640 or MEM containing antibiotics such as streptomycin and penicillin can be illustrated as media to culture hybridoma in the in vitro system.

The hybridoma developing anti-m GPCR monoclonal antibody can be produced, for instance, by immunizing a BALB/c mouse with GPCRs obtained from a mouse and the like, cell-fusing spleen cells of the immuned mouse with a mouse NS-1 cell (ATCC TIB-18) by usual methods, and then screening by the immunnofluorescence dye pattern. As the separation and the purification method of said monoclonal antibody, any conventional methods used for purification of a protein can be used and the liquid chromatography, such as affinity chromatography, is concretely exemplified.

Further, in order to develop a single stranded antibody to the above-mentioned protein and the peptide of the present invention, the preparation method of a single stranded antibody (U.S. Pat. No. 4,946,778) can be applied. Further, in order to express a humanized antibody, it is possible to use transgenic mice, other mammalian animals or the like, to isolate and identify the clones which express the protein and the peptide of the present invention with the above-mentioned antibody, and to purify its polypeptide by affinity chromatography. The antibody to the protein and the peptide of the present invention and the peptide containing their antigen epitopes are useful for elucidation of the molecular mechanism of GPCR proteins. The recombinant protein or the recombinant peptide to which these antibodies specifically bind are also included in the protein and the peptide of the present invention as described above.

Further, the function of the protein and the peptide of the present invention can be analyzed by using the fusion protein constructed by fusing the antibody, such as the above-mentioned anti-mGPCR monoclonal antibody and the like, with a fluorescent substance, for instance, such as FITC (fluorescein isothiocyanate), tetramethyl rhodamine isocyanate or the like, a radioisotope such as ¹²⁵I, ³²P, ¹⁴C, ³⁵S, ³H or the like, an labeled product with an enzyme such as alkaline phosphatase, peroxidase, β-galactosidase, phycoerythrin or the like, or a fluorescent and luminescent protein such as a green fluorescent protein (GFP). Further, the methods such as RIA, ELISA, the fluorescence antibody technique, the plaque forming cell assay, the spot test, the hemagglutination method, and the Ouchterlony method and the like can be exemplified as the immunological method for measurement.

The present invention also relates to a host cell containing an expression system which can express the protein and the peptide of the present invention. The gene which encodes the protein and the peptide of the present invention can be introduced into a host cell by a number of methods described in many standard laboratory manuals such as by Davis et al. (BASIC METHODS IN MOLECULAR BIOLOGY, 1986), and by Sambrook et al. (MOLECULAR CLONING: A LABORATORY MANUAL, 2nd Ed., Cold Spring Harbor Laboratory Press, Cold Spring Harbor, N.Y., 1989) and so on. Examples of those methods include calcium phosphate transfection, DEAE-dextran-mediated transfection, transvection, microinjection, cationic lipid-mediated transfection, electroporation, transduction, scrape loading, ballistic introduction and infection. Examples of the host cells include bacterial procaryotic cells such as Escherichia coli, Streptomyces, Bacillus subtilis, Streptococcus, Staphylococcus and the like; fungous cells such as yeast, Aspergillus and the like; insect cells such as drosophila S2, spodptera Sf9 and the like; and animal or plant cells such as L cells, CHO cells, COS cells, HeLa cells, C127 cells, BALB/c3T3 cells (including mutant strains deficient in dihydrofolate reductase, thymidine kinase or the like), BHK21 cells, HEK293 cells, Bowes melanoma cells, oocytes and the like.

As the expression system, any expression systems which can express the protein and the peptide of the present invention in the host cell can be used, and the examples include expression systems derived from chromosome, episome and virus, such as; expression systems derived from bacterial plasmid and yeast plasmid; vectors derived from papovavirus like SV40, vaccinia virus, adenovirus, chicken pox virus, pseudorabies virus, and retrovirus; and vectors derived from bacteriophage, transposon, and the combination of these, for instance, vectors derived from genetic factors of plasmid, such as cosmid or phagemid, and bacteriophage. These expression systems may contain a regulatory sequence which acts not only as a promoter but also as a controller of the expression.

The host cell containing the above-mentioned expression system, the cell membrane of said host cell, and the protein and the peptide of the present invention obtainable by the cultivation of said host cell can be used in the screening method of the present invention as hereinafter described. For example, the method of F. Pietri-Rouxel et al. (Eur. J. Biochem., 247, 1174-1179, 1997) or the like can be used as the method to obtain cell membranes, and publicly known methods including ammonium sulfate or ethanol precipitation, acid extraction, anion or cation exchange chromatography, phosphocellulose chromatography, hydrophobic-interaction chromatography, affinity chromatography, hydroxyapatite chromatography, and lectin chromatography, preferably high-speed liquid chromatography can be used to collect the protein and the peptide of the present invention from cell cultured material and then purify them. In particular, as columns used for affinity chromatography, there are columns to which the antibody to the protein and the peptide of the present invention, such as the anti-GPCR monoclonal antibody and the like, is bound, or in case that a normal peptide tag is added to the protein and the peptide of the present invention, there are columns to which materials having affinity to said peptide tag is bound. The protein and the peptide of the present invention can be obtained by using these columns. The above-mentioned purification method of the protein and the peptide of the present invention can also be applied in the peptide synthesis.

In the present invention, a non-human animal overexpressing the above-mentioned protein and the peptide of the present invention means a non-human animal which produces larger amount of the protein and the peptide of the present invention than a wild-type non-human animal does. In addition, a non human animal whose function of a gene encoding the protein and the peptide of the present invention is deficient on its chromosome means a non-human animal wherein a part or the whole of a gene encoding the protein and the peptide of the present invention on its chromosome is inactivated by gene mutation such as disruption, deficiency, substitution, etc. and function of expressing the protein and the peptide of the present invention is lost. Though specific examples of a non-human animal of the present invention include rodents and the like, such as rabbits, mice, rats etc., a non-human animal of the present invention is not limited to these animals.

A Homozygous non-human animal generated according to Mendelian ratio includes a deficient type or an overexpression type of the protein and the peptide of the present invention, and their littermate wild-type. It is possible to carry out precise comparative experiments in individual level by using the deficient type or the overexpression type and the littermate wild-type of these homozygous non-human animals at the same time. Therefore, it is preferable to use the wild-type non-human animals, in other words, those of the same species, more preferably the littermates, of the non-human animals being deficient in or overexpressing the function of a gene which encodes the protein and the peptide of the present invention on their chromosome at the same time, for example, in the screening of the present invention hereinafter described. The generating method of the non-human animals being deficient in or overexpressing the function of a gene which encodes the protein and the peptide of the present invention on their chromosome will be explained below, with examples of knockout mice and transgenic mice of GPCR proteins.

For example, a mouse deficient in the function of a gene which encodes GPCR proteins on its chromosome, in other words, a knockout mouse of GPCR proteins can be constructed as follows. A gene which encodes the above-mentioned GPCR proteins is screened by using a gene fragment obtained from mouse gene library by a method like PCR. The screened gene which encodes GPCR proteins is subcloned with a viral vector or the like and specified by DNA sequencing. A target vector is constructed by substituting the whole or a part of the gene of this clone which encodes GPCR proteins with pMC1 neo gene cassette or the like, and by introducing a diphteria toxin A fragment (DT-A) gene, a herpes simplex virus thymidine kinase (HSV-tk) gene or other such genes into 3′-terminal side.

This constructed target vector is linearized and introduced into ES cells by electroporation or the like to induce homologous recombination. The ES cells wherein homologous recombination is induced by an antibiotic such as G418, ganciclovir (GANC) or the like are selected from the homologous recombinants. It is preferable to confirm whether the selected ES cells are the objected recombinants by Southern blot analysis or the like. A chimeric mouse is constructed by microinjecting a clone of the confirmed ES cells into a blastocyst of a mouse and then transplanting the blastocyst into a recipient mouse. A heterozygous mouse can be obtained by intercrossing the chimeric mouse with a wild-type mouse, and a knockout mouse of GPCR proteins of the present invention can be constructed by intercrossing the heterozygous mice. It is possible to confirm whether a knockout mouse of GPCR proteins is constructed, for example, by isolating RNA from the mouse obtained by the above-mentioned method and examining it by Northern blot analysis or the like, or by examining the expression of the mouse by Western blot analysis or the like.

A transgenic mouse of GPCR proteins can be generated in following procedures. A transgene is constructed by fusing chicken β-actin, mouse neurofilament, SV40 or other such promoters, and rabbit β-globin, SV40 or other such poly A or introns with cDNA encoding GPCR proteins. The transgene is microinjected into the pronucleus of a fertilized egg of a mouse, and the obtained egg cell is cultured, then transplanted into the oviduct of a recipient mouse. After rearing up the recipient animal, baby mice with the above-mentioned cDNA are selected from the mice born from the recipient animal. Thus transgenic mice can be generated. The baby mouse with cDNA can be selected by extracting crude DNA from a tail or the like of a mouse, then carrying out methods like dot hybridization using the introduced gene encoding GPCR proteins as a probe, PCR method using a specific primer, and the like.

Examples of the screening method of the promoter or the suppressor of the function of the protein and the peptide of the present invention, such as GPCRs of the present invention include: a method characterized in using the above-mentioned protein and the peptide of the present invention or a cell membrane which expresses the protein and the peptide of the present invention and a test substance; a method characterized in using the above-mentioned protein and the peptide of the present invention or a cell membrane which expresses the protein and the peptide of the present invention, the G protein and a test substance; a method characterized in using a cell which expresses the protein and the peptide of the present invention and a test substance; a method characterized in using a non-human animal, such as a knockout mouse or a transgenic mouse of the protein and the peptide of the present invention and a test substance.

One example of the screening method characterized in using the above-mentioned protein and the peptide of the present invention or a cell membrane which expresses the protein and the peptide of the present invention and a test substance is a method wherein the protein and the peptide of the present invention or the protein and the peptide of the present invention expressed on the surface of a cell membrane is made to contact with a test substance, and then the binding condition of the protein and the peptide of the present invention or the protein and the peptide of the present invention expressed on the surface of a cell membrane and a test substance is measured and evaluated. One example of the method characterized in using the above-mentioned protein and the peptide of the present invention or a cell membrane which expresses the protein and the peptide of the present invention, the G protein and a test substance is a method wherein the protein and the peptide of the present invention or the protein and the peptide of the present invention expressed on the surface of the cell membrane and the G protein is made to contact with a test substance, and then the interaction of the protein and the peptide of the present invention or the protein and the peptide of the present invention expressed on the surface of the cell membrane with the G protein is measured and evaluated.

Examples of the method characterized in using a non-human animal, such as a knockout mouse or a transgenic mouse of the protein and the peptide of the present invention and a test substance include: a method wherein a cell which expresses the protein and the peptide of the present invention derived from said non-human animals is made to contact, in advance, with a test substance in vitro, and said cell which expresses the protein and the peptide of the present invention is cultivated in the presence of the G protein, and then the reaction to the G protein in the cell which expresses the protein and the peptide of the present invention is measured and evaluated; a method wherein a cell which expresses the protein and the peptide of the present invention derived from the above-mentioned non-human animals is made to contact, in advance, with the G protein in vitro, and the cell which expresses the protein and the peptide of the present invention is cultivated in the presence of a test substance, and then the reaction to the G protein, such as the binding condition to the G protein and the like, in the cell which expresses the protein and the peptide of the present invention is measured and evaluated; a method wherein a test substance is administered in advance to the above-mentioned non-human animals, and a cell which expresses the protein and the peptide of the present invention derived from the non-human animals is cultivated in the presence of the G protein, and then the reaction to the G protein in the cell which expresses the protein and the peptide of the present invention is measured and evaluated.

The promoter or the suppressor of the function or the expression of the protein and the peptide of the present invention, such as ligands of GPCR and the like, can be obtained by the above-mentioned screening methods. The medical constituent of the present invention is not limited in particular, as far as it comprises the protein and the peptide of the present invention and the promoter or the suppressor of the function or the expression of the protein and the peptide of the present invention as an active component. These medical constituents can be used for medical treatment for a patient who needs promotion of the function or the expression of the protein and the peptide of the present invention, or for a patient who needs suppression of the function or the expression of the protein and the peptide of the present invention. In addition, the diagnostic method for diseases relating to the function or the expression of the protein and the peptide of the present invention is not limited in particular, as far as it comprises a comparison of a DNA sequence encoding the GPCR protein of the present invention in a sample with the above-mentioned DNA sequence encoding the GPCR protein of the present invention. The diagnostic probe for diseases relating to the function or the expression of the protein and the peptide of the present invention is not limited in particular, as far as it comprises the whole or a part of the above-mentioned antisense chain of DNA or RNA encoding the GPCR protein of the present invention. The diagnostic drug for diseases relating to the function or the expression of the protein and the peptide of the present invention is not limited in particular, as far as it contains the above-mentioned diagnostic probe or the above-mentioned antibodies.

The present invention is now explained in more detail with examples, but the scope of the present invention is not limited to these examples.

GPCR genes other than odorant and taste receptors so far reported were examined whether they contained introns or not. 95 GPCR genes were confirmed not to contain introns and 63 GPCR genes were confirmed to contain introns. Therefore, about 60% of GPCR genes are predicted that they have no introns. In addition, the presence of introns in human odorant and taste receptors has not yet been reported. Thus, GPCRs without introns were searched for as hereinafter described.

[Extraction of Putative GPCR Genes from the Human Genome Data]

Open reading frames (ORF), which could be the candidates of GPCR genes, were extracted from the human genome data as of Aug. 18, 2000, and analyzed by SOSUI. As this human genome data used contains the same undetermined sequences duplicately, it consists of about 4.5G base pairs while the entire human genome consists of about 3G base pairs. About 210,000 ORFs consisting of DNA encoding 200-1500 amino acid residues were extracted from the human genome data, and among them, 130,000 ORFs were obtained by eliminating ORFs considered to be derived from repeated sequences of DNA, ORFs comprising substantial undetermined amino acids, or ORFs in which any single amino acid residue comprising more than 20% of the whole sequence. Prior to analyze these 130,000 ORFs by SOSUI, the efficiency of SOSUI analysis was examined.

[Efficiency of SOSUI Analysis]

Analysis by SOSUI has a possibility of interpreting a signal peptide of GPCRs as a transmembrane segment, or not recognizing seventh transmembrane segment as a transmembrane segment due to its tendency of low hydrophobicity in the structure of GPCRs. Therefore, the efficiency of SOSUI in searching for GPCRs was first checked by analyzing the amino acid sequences of known GPCRs by SOSUI. 207 GPCRs other than odorant and taste receptors so far reported were analyzed by SOSUI, and the analysis result was that 95% of them, 197 GPCRs, had 6 to 8 transmembrane segments. 43 odorant receptors were also analyzed in the same way, and the analysis result that 77% of them, 33 GPCRs, had 6 to 8 transmembrane segments was obtained. Therefore, the prediction ability of SOSUI was considered to be about 95% for GPCRs other than odorant and taste receptors, and about 77% for odorant receptors. The above-mentioned 207 GPCRs other than taste and odorant receptors so far reported which had been downloaded from the website http://www.gpcr.org/7tm/seq/dna.html, and the genes of odorant receptors which had been downloaded from the website http://ycmi.med.yale.edu/senselab/ORDB were used.

[Homology (BLAST) Search]

After SOSUI analysis of the above-mentioned 130,000 ORFs, 1150 ORFs having 6 to 8 transmembrane segments were obtained. The sequences of 1150 ORFs of the obtained GPCR candidates were referred to the “nr” data base as of Aug. 20, 2000 using BLAST and analyzed for their homology to known GPCRs. As a result, 522 ORFs homologous to known GPCRs were obtained. Then, by removing duplicated ORFs from the above-mentioned sequences, 203 known odorant receptor genes, 11 known taste receptor genes, and 64 registered GPCR genes other than odorant and taste receptors were able to be identified. On the other hand, novel GPCR genes other than odorant and taste receptors, novel GPCR genes of odorant receptors, or novel GPCR genes of taste receptors, all of which have homology to known GPCR genes were obtained by BLAST search.

[Amino Acid Sequence Analysis]

The amino acid sequences of the above-mentioned novel GPCR genes obtained by BLAST search were analyzed. As to the amino acid sequence analysis, SPORT was used to identify signal sequences in addition to the above-mentioned SOSUI, and then it is examined if lengths of the loop between the transmembrane segments were appropriate or not. As to the gene sequences, in order to judge the possibility that ORFs could be pseudogenes, the existence of TATAbox or CpGisland in the upstream of ORFs was examined. Consequently, 51 ORFs presumed to be novel GPCRs other than odorant and taste receptors, 281 ORFs presumed to be novel GPCRs of odorant receptors, 218 of which contain sequences specific to odorant receptors, and 15 ORFs presumed to be novel GPCRs of taste receptors were confirmed. The base sequences of the above-mentioned 51 novel GPCRs other than odorant and taste receptors are represented in Seq. ID No. 2n-1 (n=any one of integral numbers 1 to 51), and the amino acid sequences of the novel GPCR proteins encoded by these 51 novel GPCR genes are represented in Seq. ID No. 2n (n=any one of integral numbers 1 to 51). The base sequences of novel GPCR genes of 281 odorant receptors are represented in Seq. ID No. 2n-1 (n=any one of integral numbers 52 to 332), and the amino acid sequences of the novel GPCR proteins of these 281 odorant receptors are represented in Seq. ID No. 2n (n=any one of integral numbers 52 to 332). The base sequences of novel GPCR genes of 15 taste receptors are represented in Seq. ID No. 2n-1 (n=any one of integral numbers 333 to 347), and the amino acid sequences of the novel GPCR proteins of these 15 taste receptors are represented in Seq. ID No. 2n (n=any one of integral numbers 333 to 347).

The proteins having the highest homology to the transmembrane segments (TM) of the above-mentioned 347 novel GPCR proteins at the amino acid level are hereinafter described. A figure following to TM represents the number of the transmembrane segment, followed by a numeric range which shows the number of amino acid residues which consist in the transmembrane segment in the sequence listing, (P) following to the number of the amino acid residues in the sequence listing shows extremely clear characteristic as a transmembrane segment, and (S) shows weaker characteristic as a transmembrane segment. Further, figures following to the name of the proteins having the highest homology at the amino acid level represent the accession number, and the counts of the amino acids which completely correspond and their ratios (%) to all amino acids are also shown. Further, when expression sites were able to be confirmed by RT-PCR, the expression sites are described after that.

Seq. ID. No. 2 [TM1;26-50(P), TM2;59-83(P), TM3;92-116(P), TM4;130-154(P), TM5;168-192(P), TM6;203-227(P), TM7;241-265(P)]

retinoic acid induced 3 (putative G protein-coupled receptor)(AF095448);138/290 (47%)

Spleen, lung, heart, liver, kidney, pancreas, small intestine, colon

Seq. ID. No. 4 [TM1;17-40(S), TM2;66-88(P), TM3;99-123(S), TM4;138-162(S), TM5;222-246(P), TM6;256-280(P)] hypothetical protein P1.11659_{—}3-human(AC004472);302/307 (98%)

Heart, colon

Seq. ID. No. 6 [TM1;1-21(S), TM2;92-116(P), TM3;126-148(P), TM4;177-197(S), TM5;207-231(S), TM6;255-279(P), TM7;298-322(P)]

putative chemokine receptor; GTP-binding protein(PROTEIN-COUPLED RECEPTOR HM74)(D10923);122/294 (41%)

Seq. ID. No. 8 [TM1;18-42(P), TM2;49-73(S), TM3;100-120(S), TM4:138-162(P), TM5;178-202(S), TM6;219-243(P)]

putative chemokine receptor; GTP-binding protein(PROTEIN-COUPLED RECEPTOR HM74)(D10923);178/341 (52%)

Seq. ID. No. 10 [TM1;31-55(P), TM2;65-89(P), TM3;99-123(S), TM4;154-178(P), TM5;194-218(P), TM6;242-266(S), TM7;274-298(P)]

FML1_GORGO FMLP-RELATED RECEPTOR I (X97738);90/324 (27%) Seq. ID. No. 12 [TM1;24-48(P), TM2;69-90(S), TM3;96-120(S), TM4;135-159(P), TM5;186-210(P), TM6;231-251(P), TM7;277-301(P)]

P2Y5_CHICK P2Y PURINOCEPTOR 5 (P2Y5) (protein-coupled receptor)(L06109);113/284 (39%)

Seq. ID. No. 14 [TM1;37-61(P), TM2;80-102(S), TM3;118-142(P), TM4;150-174(P), TM5;200-222(P), TM6;240-264(P)]

purinergic receptor P2Y, G-protein coupled 1 (U22829);109/299 (36%)

Brain, spleen, lung, liver, colon

Seq. ID. No. 16 [TM1;27-51(P), TM2;69-93(P), TM3;103-124(S), TM4;144-168(P), TM5;185-209(P), TM6;223-247(P), TM7;267-291(S)]

MAS1 oncogene (J03823);111/294 (37%)

Heart, small intestine, colon

Seq. ID. No. 18 [TM1;8-32(P), TM2;61-85(S), TM3;92-116(S), TM4;154-178(P), TM5;196-220(P), TM6;250-274(P)]

G-protein coupled receptor GPR34 (AF039686);77/323 (23%) Brain, spleen, lung, heart, lung, kidney, pancreas, stomach, small intestine, colon

Seq. ID. No. 20 [TM1;5-23(S), TM2;50-74(P), TM3;87-111(S), TM4;123-147(P), TM5;165-189(P), TM6;211-235(P)]

5-hydroxytryptamine (serotonin) receptor ID (AB041379);72/272 (26%)

Pancreas

Seq. ID. No. 22 [TM1;2-24(P), TM2;29-53(P), TM3;64-88(S), TM4;94-117(P), TM5;213-237(P), TM6;297-321(S)]

CG2114 gene product (AE003476);71/302 (23%)

Stomach, small intestine

Seq. ID. No. 24 [TM1;37-61(P), TM2;76-100(S), TM3;113-137(P), TM4;151-175(P), TM5;189-213(P), TM6;250-274(P), TM7;290-310(S)]

sphingosine 1-phosphate receptor Edg-8 (AF223649);343/397 (86%)

Seq. ID. No. 26 [TM1;25-49(P), TM2;58-82(S), TM3;97-121(P),

TM4;138-162(P), TM5;184-208(P), TM6;233-254(P), TM7;274-293(S)]

gene product; putative G-protein-coupled receptor; G protein coupled receptor for UDP-glucose (D13626);149/314 (47%) Brain, spleen, heart, stomach, colon

Seq. ID. No. 28 [TM1;39-63(P), TM2;72-96(S), TM3;148-170(P), TM4;202-225(P), TM5;248-272(P), TM6;286-310(S)]

GALS_MOUSE GALANIN RECEPTOR TYPE 2 (AF042784);109/299 (36%)

Pancreas

Seq. ID. No. 30 [TM1;43-67(P), TM2;79-103(P), TM3;113-137(P), TM4;154-174(S), TM5;206-230(P), TM6;287-311(P), TM7;324-345(S)]

PROBABLE G PROTEIN-COUPLED RECEPTOR (D43633);179/429 (41%) Seq. ID. No. 32 [TM1;135-157(S), TM2;161-184(P), TM3;191-215(P), TM4;230-254(P), TM5;266-290(P), TM6;318-342(P)] seven transmembrane receptor (AB019120);71/302 (23%)

Seq. ID. No. 34 [TM1;43-67(P), TM2;79-103(P), TM3;113-137(P), TM4;154-178(S), TM5;206-230(P), TM6;280-302(P), TM7;312-336(P)]

PROBABLE G PROTEIN-COUPLED RECEPTOR (D43633);125/312 (40%) Seq. ID. No. 36 [TM1;38-62(P), TM2;89-113(S), TM3;118-142(P), TM4;164-188(P), TM5;208-232(P), TM6;248-272(S), TM7;276-298(S)]

G-protein coupled receptor SALPR; somatostatin and angiotensin-like (G-protein coupled receptor SALPR) (D88437);141/322 (43%)

Kidney, small intestine

Seq. ID. No. 38 [TM1;7-31(P), TM2;42-66(S), TM3;79-103(S), TM4;120-144(P), TM5;159-183(P), TM6;224-248(P), TM7;264-288(S)]

DOPI_DROME DOPAMINE RECEPTOR 1 PRECURSOR (D-DOP1) (X77234);95/350 (27%)

Seq. ID. No. 40 [TM1;3-27(S), TM2;36-60(P), TM3;64-87(S), TM4;94-116(S), TM5;213-231(S), TM6;251-273(P)] cadherin EGF LAG seven-pass G-type receptor (AF031572);40/112 (35%)

Seq. ID. No. 42 [TM1;27-51(S), TM2;103-122(S), TM3;139-160(P), TM4;180-204(S), TM5;221-245(P), TM6;263-281(S)]

RTA_RAT PROBABLE G PROTEIN-COUPLED RECEPTOR RTA (M35297);120/332 (36%)

Seq. ID. No. 44 [TM1;28-52(P), TM2;63-87(P), TM3;95-117(P), TM4;141-165(P), TM5;179-203(P), TM6;216-240(P), TM7;252-276(S)]

MASI oncogene (J03823);108/283 (38%)

Spleen, lung, colon

Seq. ID. No. 46 [TM1;18-42(S), TM2;96-120(P), TM3;140-164(P), TM4;178-202(P), TM5;318-342(P), TM6;353-374(S)] (AE003422) EG:22E5.11 gene product (AE003422);98/364. (26%)

Lung

Seq. ID. No. 48 [TM1;12-36(P), TM2;47-71(S), TM3;97-121(P), TM4;133-157(S), TM5;183-207(P), TM6;226-250(P)]

frizzled 6 (AB012911);286/314 (91%)

Seq. ID. No. 50 [TM1;16-40(P), TM2;45-69(S), TM3;80-104(P), TM4;116-138(S), TM5;164-188(S), TM6;278-302(P), TM7;354-378(P)]

unnamed protein product (AK000922);286/370 (77%)

Seq. ID. No. 52 [TM1;64-88(P), TM2;98-120(P), TM3;122-146(S), TM4;172-196(P), TM5;213-237(S), TM6;253-277(P), TM7;284-308(P)]

putative pheromone receptor V2R2 (AF053989);250/327 (76%)

Nose

Seq. ID. No.54 [TM1;84-106(P), TM2;110-132(P), TM3;136-158(P), TM4;173-194(P), TM5;200-224(P), TM6;230-251(P), TM7;267-291(P)]

insulin-like growth factor I, brain-specific—Thai catfish (A53697);28/88 (31%)

Seq. ID. No.56 [TM1;90-114(S), TM2;117-140(S), TM3;151-168(S), TM4;174-198(P), TM5;209-233(S), TM6;238-262(P)]

hypothetical protein T06E4.7—Caenorhabditis elegans (Z70756);25/67 (37%)

Lung

Seq. ID. No. 58 [TM1;40-64(P), TM2;71-95(S), TM3;124-148(P), TM4;153-177(P), TM5;204-228(P), TM6;243-265(P), TM7;283-307(S)]

cysteinyl leukotriene receptor 1 (AF119711);114/298 (38%) Seq. ID. No. 60 [TM1;16-38(P), TM2;51-73(P), TM3;91-113(P), TM4;133-155(S), TM5;178-199(P), TM6;271-293(S)]

G protein-coupled seven-transmembrane receptor(D43633);117/365 (32%)

Seq. ID. No. 62 [TM1;17-39(S), TM2;67-89(P), TM3;101-123(S), TM4;135-157(P), TM5;178-196(S), TM6;202-221(S)]

allatostatin receptor(AF163775);43/154 (27%)

Lung, heart, pancreas, small intestine

Seq. ID. No. 64 [TM1;9-31(S), TM2;48-70(S), TM3;92-114(S), TM4;207-229(S), TM5;269-290(P), TM6;315-337(P), TM7;348-370(P)]

pheromone receptor VN3(U36895);84/277 (30%)

Nose

Seq. ID. No. 66 [TM1;118-140(P), TM2;153-175(S), TM3;199-221(S), TM4;236-258(P), TM5;281-303(P), TM6;326-348(P), TM7;362-384(P)]

G protein-coupled receptor LGR5(AF061444);120/269 (44%) Seq. ID. No. 68 [TM1;33-54(S), TM2;97-119(P), TM3;168-190(S), TM4;264-286(P), TM5;291-313(S), TM6;405-427(P)]

interleukin 9 receptor precursor(L39064);30/105 (28%)

Seq. ID. No. 70 [TM1;5-27(P), TM2;55-76(S), TM3;85-107(P), TM4;126-148(S), TM5;164-186(P), TM6;267-289(P), TM7;304-326(S)]

pheromone receptor VN6(U36898);99/310 (31%)

Nose

Seq. ID. No. 72 [TM1;1-21(S), TM2;27-43(S), TM3;51-73(S), TM4;92-114(S), TM5;131-153(S), TM6;183-205(P)]

pheromone receptor 1(Y12725);64/155 (41%)

Nose

Seq. ID. No. 74 [TM1;20-42(P), TM2;50-72(S), TM3;84-106(P), TM4;123-144(P), TM5;167-189(S), TM6;228-250(P), TM7;259-281(S)]

putative G-Protein coupled receptor, EDG6(AJ000479);81/268 (30%)

Skeletal muscle

Seq. ID. No. 76 [TM1;21-43(P), TM2;55-77(P), TM3;100-122(S), TM4;136-158(P), TM5;181-203(P), TM6;227-247(P), TM7;270-272(S)]

putative purinergic receptor P2Y10(AF000545);148/292 (50%) Seq. ID. No. 78 [TM1;24-46(P), TM2;57-79(P), TM3;98-120(P), TM4;136-158(P), TM5;189-211(P), TM6;234-255(P), TM7;280-300(S)]

KIAA0001 gene product(D13626);140/295 (47%)

Brain, thymus, lung, kidney, stomach, colon

Seq. ID. No. 80 [TM1;1-23(P), TM2;36-58(S), TM3;249-271(P), TM4;296-318(P), TM5;332-354(P), TM6;368-390(P), TM7;412-434(P), TM8;458-480(S)]

seven transmembrane receptor(AB019120);191/487 (39%) Seq. ID. No. 82 [TM1;5-25(S), TM2;80-97(S), TM3;103-124(P), TM4;133-155(P), TM5;165-187(S), TM6;217-239(P)]

unnamed protein product(AK000922);304/357 (85%) Seq. ID. No. 84 [TM1;16-34(S), TM2;216-238(P), TM3;247-269(S), TM4;285-307(S), TM5;367-389(P), TM6;403-425(P)]

seven transmembrane receptor(AB019120);95/326 (29%)

Seq. ID. No. 86 [TM1;1-23(S), TM2;41-63(S), TM3;95-117(P), TM4;128-150(P), TM5;157-179(P), TM6;190-212(S), TM7;216-238(P), TM8;260-282(P)]

neuropeptide Y receptor Y6(U58367);23/88 (26%)

Seq. ID. No. 88 [TM1;14-36(S), TM2;55-77(P), TM3;94-116(P), TM4;126-147(P), TM5;185-207(P), TM6;215-237(S)]

orphan G protein-coupled receptor(AF045764);199/236 (84%)

Seq. ID. No. 90 [TM1;31-53(P), TM2;69-91(P), TM3;110-132(S), TM4;195-217(P), TM5;400-422(P), TM6;434-456(P)]

G-protein coupled receptor RE2(AF091890);63/192 (32%)

Seq. ID. No. 92 [TM1;1-23(P), TM2;36-58(P), TM3;99-121(P), TM4;135-157(S), TM5;165-187(P), TM6;189-211(S), TM7;339-361(P), TM8;379-401(S)]

Frizzled-1 protein homolog(T42210);27/82 (32%)

Seq. ID. No. 94 [TM1;24-46(P), TM2;83-105(P), TM3;122-143(P), TM4;166-188(S), TM5;227-249(P), TM6;258-280(S)]

putative G-Protein coupled receptor(AJ000479);76/268 (28%)

Seq. ID. No.96 [TM1;22-44(P), TM2;113-135(P), TM3;159-181(P), TM4;203-225(S), TM5;228-250(P), TM6;272-294(S)]

Frizzled-10(AB027464);23/96 (23%)

Seq. ID. No. 98 [TM1;11-33(S), TM2;41-63(S), TM3;71-93(S), TM4;96-118(P), TM5;122-144(S), TM6;146-165(S)]

beta3-adrenergic receptor(AF109930);39/120 (32%)

Seq. ID. No. 100 [TM1;8-29(P), TM2;31-52(P), TM3;55-73(P), TM4;79-100(P), TM5;110-132(S), TM6;147-169(P), TM7;177-199(P), TM8;201-222(P)]

putative pheromone receptor V2R1(AF053985);51/219 (23%)

Seq. ID. No. 102 [TM1;6-28(P), TM2;60-82(S), TM3;90-111(S), TM4;127-149(P), TM5;181-203(S), TM6;237-259(P)]

pheromone receptor VN6(U36898);79/236 (33%)

Seq. ID. No. 104 [TM1;5-27(S), TM2;41-63(P), TM3;82-104(S), TM4;106-128(P), TM5;153-175(S), TM6;191-213(P), TM7;235-257(P), TM8;289-311(S)]

odorant receptor S19(AF121976);106/228 (46%)

Seq. ID. No. 106 [TM1;1-23(S), TM2;44-66(S), TM3;85-106(P), TM4;149-171(P), TM5;183-205(S), TM6;217-238(S)]

odorant receptor S18(AF121975);159/222 (71%)

Seq. ID. No. 108 [TM1;3-25(P), TM2;96-118(S), TM3;120-142(S), TM4;149-171(P), TM5;182-204(P), TM6;211-231(P)]

odorant receptor A16(AB030896);122/244 (50%)

Seq. ID. No. 110 [TM1;31-53(P), TM2;64-86(S), TM3;100-122(S), TM4;138-160(P), TM5;171-193(S), TM6;199-221(P)]

olfactory receptor-like protein COR3′beta(L17432);106/217 (48%)

Seq. ID. No. 112 [TM1;30-52(P), TM2;65-87(P), TM3;105-127(S), TM4;134-156(S), TM5;203-225(P), TM6;243-265(P), TM7;272-294(S)]

odorant receptor S18(AF121975);184/303 (60%)

Seq. ID. No. 114 [TM1;29-51(P), TM2;59-81(S), TM3;99-121(S), TM4;144-166(P), TM5;179-201(S), TM6;204-225(P), TM7;242-264(P), TM8;269-291(S)]

odorant receptor MOR83(AB030894);155/306 (50%)

Seq. ID. No. 116 [TM1;4-26(S), TM2;32-53(P), TM3;65-87(P), TM4;99-121(S), TM5;123-144(P), TM6;162-184(S), TM7;189-211(P)]

odorant receptor MOR83(AB030894);121/226 (53%)

Seq. ID. No. 118 [TM1;26-48(P), TM2;61-83(S), TM3;98-120(S), TM4;141-163(S), TM5;204-225(P), TM6;239-261(P)]

odorant receptor MOR83(AB030894);156/303 (51%)

Seq. ID. No. 120 [TM1;25-47(P), TM2;57-79(S), TM3;88-109(S), TM4;131-153(P), TM5;201-223(P), TM6;242-264(S)]

CfOLF1(U53679);107/235 (45%)

Seq. ID. No. 122 [TM1;29-51(S), TM2;83-105(P), TM3;114-136(P), TM4;149-171(S), TM5;192-214(S), TM6;259-281(P), TM7;294-316(P)]

odorant receptor S51(AF121981);127/159 (79%)

Seq. ID. No. 124 [TM1;35-57(P), TM2;68-90(P), TM3;96-118(S), TM4;147-169(S), TM5;210-232(P), TM6;246-268(P), TM7;278-300(S)]

MOR 3Beta1(AF133300);119/308 (38%)

Seq. ID. No. 126 [TM1;15-37(S), TM2;64-86(P), TM3;93-115(S), TM4;141-163(S), TM5;206-228(P), TM6;237-259(P)]

odorant receptor S51(AF121981);85/128 (66%)

Seq. ID. No. 128 [TM1;6-28(S), TM2;32-53(P), TM3;59-81(S), TM4;97-119(S), TM5;145-167(P), TM6;205-226(P), TM7;236-258(P), TM8;268-290(S)]

similar to rat olfactory receptor OR18(AC004908);209/311 (67%)

Seq. ID. No. 130 [TM1;1-23(S), TM2;28-50(P), TM3;63-85(S), TM4;142-163(P), TM5;203-225(P), TM6;238-260(P), TM7;271-293(P)]

olfactory receptor-like protein COR3′beta(L17432);147/299 (49%)

Seq. ID. No. 132 [TM1;31-53(S), TM2;63-85(S), TM3;99-121(S), TM4;149-171(P), TM5;205-227(P), TM6;244-266(S), TM7;273-295(P)]

putative G-protein coupled receptor RAlc(AF079864);142/299 (47%)

Seq. ID. No. 134 [TM1;1-23(S), TM2;26-48(S), TM3;51-73(S), TM4;85-107(S), TM5;151-172(P), TM6;182-204(P), TM7;212-234(S)]

odorant receptor S19(AF121976);142/246 (57%)

Seq. ID. No. 136 [TM1;11-33(S), TM2;37-59(P), TM3;71-93(P), TM4;107-129(P), TM5;151-173(S), TM6;207-229(P), TM7;249-271(P)]

olfactory receptor-like protein COR3′beta(L17432);134/306 (43%)

Seq. ID. No. 138 [TM1;11-32(S), TM2;49-71(S), TM3;91-113(P), TM4;128-150(S), TM5;170-192(P), TM6;231-253(P), TM7;268-290(S), TM8;312-334(S)]

olfactory receptor-like protein COR3′beta(L17432);149/313 (47%)

Seq. ID. No. 140 [TM1;1-23(S), TM2;30-52(P), TM3;67-89(S), TM4;102-124(S), TM5;137-159(S), TM6;202-224(P), TM7;239-261(P), TM8;271-293(P)]

olfactory receptor-like protein COR3′beta(L17432);156/298 (52%)

Seq. ID. No. 142 [TM1;2-24(S), TM2;33-55(P), TM3;69-91(P), TM4;106-128(S), TM5;142-164(S), TM6;208-230(P), TM7;244-266(S), TM8;276-298(S)]

putative G-protein coupled receptor RAlc(AF079864);171/305 (56%)

Seq. ID. No. 144 [TM1;31-53(P), TM2;65-87(P), TM3;141-163(S), TM4;183-205(P), TM5;212-234(P), TM6;240-262(P), TM7;273-295(S)]

olfactory receptor 64(AF071080);161/302 (53%)

Seq. ID. No. 146 [TM1;6-28(P), TM2;42-64(S), TM3;87-109(P), TM4;144-166(P), TM5;183-205(P), TM6;217-237(S)]

olfactory receptor-like protein COR3′beta(L17432);129/249 (51%)

Seq. ID. No. 148 [TM1;1-18(P), TM2;43-65(S), TM3;67-88(S), TM4;99-121(S), TM5;136-158(S), TM6;175-197(P)]

putative G-protein coupled receptor RAlc(AF079864);73/151 (48%)

Seq. ID. No. 150 [TM1;31-53(P), TM2;66-88(P), TM3;104-126(S), TM4;141-163(S), TM5;205-227(P), TM6;246-268(S), TM7;274-296(S)]

odorant receptor S18(AF121975);142/308 (46%)

Seq. ID. No. 152 [TM1;6-26(S), TM2;29-51(P), TM3;53-75(P), TM4;97-119(S), TM5;133-155(S), TM6;202-224(P), TM7;237-259(P), TM8;269-291(S)]

putative G-protein coupled receptor RAlc(AF079864);298/318 (93%)

Seq. ID. No. 154 [TM1;1-23(S), TM2;29-51(P), TM3;67-89(P), TM4;104-126(S), TM5;145-166(S), TM6;206-228(P), TM7;244-266(S), TM8;277-298(S)]

putative G-protein coupled receptor RAlc(AF079864);157/301 (52%)

Seq. ID. No. 156 [TM1;18-40(P), TM2;44-65(P), TM3;69-91(S), TM4;115-137(P), TM5;154-176(P), TM6;214-236(P), TM7;255-277(P), TM8;282-304(S)]

putative G-protein coupled receptor RAlc(AF079864);184/307 (59%)

Seq. ID. No. 158 [TM1;30-52(P), TM2;58-80(S), TM3;83-105(S), TM4;204-226(P), TM5;240-262(P), TM6;274-296(S)]

putative G-protein coupled receptor RAlc(AF079864);180/299 (60%)

Seq. ID. No. 160 [TM1;2-24(S), TM2;30-52(P), TM3;64-86(S), TM4;100-122(S), TM5;141-163(S), TM6;204-226(P), TM7;273-295(S)]

odorant receptor S18(AF121975);164/303 (54%)

Seq. ID. No. 162 [TM1;30-52(P), TM2;64-86(P), TM3;102-124(S), TM4;142-164(S), TM5;205-227(P), TM6;241-263(S), TM7;273-295(S)]

odorant receptor S18(AF121975);166/305 (54%)

Seq. ID. No. 164 [TM1;33-55(P), TM2;67-89(P), TM3;104-126(S), TM4;146-168(P), TM5;214-236(S), TM6;246-268(P), TM7;276-298(S)]

odorant receptor S19(AF121976);140/304 (46%)

Seq. ID. No. 166 [TM1;14-36(S), TM2;42-64(P), TM3;78-100(S), TM4;115-137(P), TM5;153-174(S), TM6;217-239(P)]

odorant receptor S18(AF121975);209/248 (84%)

Seq. ID. No. 168 [TM1;36-58(P), TM2;68-90(P), TM3;103-125(P), TM4;146-168(S), TM5;208-230(P), TM6;245-267(P), TM7;275-297(S)]

odorant receptor S18(AF121975);123/298 (41%)

Seq. ID. No. 170 [TM1;31-53(S), TM2;63-85(P), TM3;100-122(S), TM4;140-162(S), TM5;204-226(P), TM6;239-260(P)]

MOR 3′Beta1(AF133300);103/259 (39%)

Seq. ID. No. 172 [TM1;29-51(P), TM2;63-85(P), TM3;99-121(S), TM4;143-165(S), TM5;177-199(S), TM6;204-226(S)]

putative G-protein coupled receptor RAlc(AF079864);112/247 (45%)

Seq. ID. No. 174 [TM1;10-32(P), TM2;44-66(P), TM3;73-93(S), TM4;128-150(S), TM5;158-179(S), TM6;181-203(P)]

olfactory receptor(AF179805);27/101 (26%)

Seq. ID. No. 176 [TM1;5-27(P), TM2;46-68(S), TM3;85-107(S), TM4;123-145(S), TM5;154-176(P), TM6;182-204(S), TM7;216-237(S)]

odorant receptor S18(AF121975);149/250 (59%)

Seq. ID. No. 178 [TM1;1-23(S), TM2;28-50(S), TM3;96-118(S), TM4;134-156(S), TM5;196-218(P), TM6;238-260(P), TM7;269-291(S)]

putative G-protein coupled receptor RAlc(AF079864);172/307 (56%)

Seq. ID. No. 180 [TM1;6-28(S), TM2;40-62(P), TM3;72-94(S), TM4;111-133(S), TM5;158-180(P), TM6;214-236(P), TM7;252-274(P), TM8;283-305(S)]

olfactory receptor-like protein COR3′beta(L17432);167/304 (54%)

Seq. ID. No. 182 [TM1;29-51(S), TM2;65-87(S), TM3;108-130(S), TM4;144-165(P), TM5;201-223(P), TM6;240-262(P), TM7;271-293(S)]

olfactory receptor-like protein COR3′beta(L17432);153/296 (51%)

Seq. ID. No. 184 [TM1;2-24(S), TM2;30-52(P), TM3;64-86(S), TM4;105-127(S), TM5;204-226(P), TM6;240-262(P), TM7;274-296(S)]

olfactory receptor-like protein COR3′beta(L17432);150/303 (49%)

Seq. ID. No. 186 [TM1;4-26(S), TM2;35-57(P), TM3;72-94(S), TM4;115-137(S), TM5;151-172(S), TM6;191-213(P), TM7;249-271(S), TM8;281-303(S)]

MOR 3′Beta1(AF133300);157/298 (52%)

Seq. ID. No. 188 [TM1;36-58(P), TM2;74-96(P), TM3;108-130(P), TM4;149-171(S), TM5;209-231(P), TM6;260-282(P)]

MOR 3′Beta1(AF133300);123/307 (40%)

Seq. ID. No. 190 [TM1;30-52(S), TM2;67-89(S), TM3;103-125(S), TM4;205-227(P), TM5;242-264(S), TM6;269-291(S)]

MOR 3′Beta1(AF133300);172/307 (56%)

Seq. ID. No. 192 [TM1;31-53(P), TM2;56-78(S), TM3;81-103(S), TM4;107-129(S), TM5;145-167(P), TM6;184-206(P)]

putative G-protein coupled receptor RAlc(AF079864);98/210 (46%)

Seq. ID. No. 194 [TM1;34-56(P), TM2;71-93(P), TM3;108-130(S), TM4;144-166(P), TM5;191-212(P), TM6;224-246(P), TM7;251-273(S)]

odorant receptor 5.24(AF158963);168/328 (51%)

Seq. ID. No. 196 [TM1;8-30(S), TM2;53-75(P), TM3;105-127(P), TM4;139-161(S), TM5;167-189(P), TM6;200-222(P), TM7;231-253(P)]

olfactory receptor OR18(S29710);189/268 (70%)

Seq. ID. No. 198 [TM1;28-50(P), TM2;84-106(S), TM3;117-139(S), TM4;145-166(S), TM5;185-207(P), TM6;213-235(S), TM7;243-265(P), TM8;274-296(S)]

odorant receptor S19(AF121976);192/299 (64%)

Seq. ID. No. 200 [TM1;7-29(S), TM2;35-57(S), TM3;64-86(S), TM4;95-117(S), TM5;141-163(S), TM6;200-221(P)]

olfactory receptor I7-[(AF095725);95/96 (98%)

Seq. ID. No. 202 [TM1;26-48(P), TM2;62-84(S), TM3;96-118(P), TM4;141-163(S), TM5;204-225(P), TM6;239-261(P), TM7;268-290(S)]

odorant receptor MOR83(AB030894);159/301 (52%)

Seq. ID. No. 204 [TM1;31-53(P), TM2;65-87(S), TM3;104-126(S), TM4;205-227(P), TM5;243-265(S), TM6;269-291(S)]

odorant receptor S18(AF121975);163/310 (52%)

Seq. ID. No. 206 [TM1;1-23(S), TM2;27-49(P), TM3;65-87(S), TM4;108-130(S), TM5;205-227(P), TM6;242-264(S), TM7;268-290(S)]

olfactory receptor 67(AF133300);158/309 (51%)

Seq. ID. No. 208 [TM1;32-54(P), TM2;66-88(P), TM3;98-120(S), TM4;137-159(S), TM5;210-231(P), TM6;243-265(P)]

odorant receptor S51(AF121981);112/159 (70%)

Seq. ID. No. 210 [TM1;30-52(P), TM2;100-122(P), TM3;127-149(P), TM4;153-175(P), TM5;209-231(P), TM6;243-265(S), TM7;274-296(S)]

odorant receptor S46(AF121979);198/307 (64%)

Seq. ID. No. 212 [TM1;34-56(P), TM2;104-126(P), TM3;149-171(P), TM4;210-232(P), TM5;247-269(S), TM6;276-298(S)]

odorant receptor S46(AF121979);209/307 (68%)

Seq. ID. No. 214 [TM1; 3-25(S), TM2;31-53(P), TM3;106-128(P), TM4;132-154(S), TM5;156-178(S), TM6;203-225(P), TM7;242-264(P), TM8;271-293(S)]

odorant receptor S46(AF121979);174/306 (56%)

Seq. ID. No.216 [TM1;36-58(P), TM2;101-123(P), TM3;141-163(P), TM4;181-203(P), TM5;206-227(P), TM6;270-292(S)]

odorant receptor S46(AF121979);178/307 (57%)

Seq. ID. No. 218 [TM1;26-48(P), TM2;65-87(S), TM3;104-126(S), TM4;142-164(S), TM5;204-226(P), TM6;240-262(P), TM7;276-297(S)]

olfactory receptor-like protein COR3′beta(L17432);159/301 (52%)

Seq. ID. No. 220 [TM1;21-43(P), TM2;84-106(S), TM3;116-138(S), TM4;145-167(P), TM5;182-204(S), TM6;210-232(P), TM7;240-262(S), TM8;272-294(S)]

odorant receptor S46(AF121979);192/310 (61%)

Seq. ID. No. 222 [TM1;27-49(S), TM2;75-97(S), TM3;113-135(P), TM4;140-162(P), TM5;169-190(S), TM6;203-225(S)]

olfactory receptor(AJ133430);190/247 (76%)

Seq. ID. No. 224 [TM1;1-20(S), TM2;28-50(P), TM3;61-83(S), TM4;92-114(S), TM5;133-155(P), TM6;195-217(P)]

HUMAN OLFACTORY RECEPTOR 5I1(Q13606);95/214 (44%)

Seq. ID. No. 226 [TM1;27-49(P), TM2;58-80(S), TM3;93-115(S), TM4;143-165(S), TM5;178-200(S), TM6;206-227(P), TM7;241-263(P), TM8;268-290(S)]

odorant receptor MOR83(AB030894);171/307 (55%)

Seq. ID. No. 228 [TM1;25-47(P), TM2;55-77(S), TM3;100-122(S), TM4;143-165(P), TM5;170-192(S), TM6;205-226(P), TM7;241-263(P)]

odorant receptor MOR83(AB030894);158/308 (51%)

Seq. ID. No. 230 [TM1;1-23(P), TM2;27-49(S), TM3;81-103(P), TM4;143-165(P), TM5;184-206(P), TM6;214-236(P)]

olfactory receptor [Homo sapiens] (AJ003147);154/249 (61%)

Seq. ID. No. 232 [TM1;27-49(P), TM2;63-85(P), TM3;103-125(S), TM4;143-165(S), TM5;204-226(P), TM6;238-259(S), TM7;273-295(S)]

olfactory receptor P2 [Mus musculus ] (AF247657);194/302 (64%)

Seq. ID. No.234 [TM1;34-55(S), TM2;104-126(P), TM3;148-170(S), TM4;204-226(P), TM5;242-264(S), TM6;273-294(S)]

HsOLF1 [Homo sapiens] (U56420);153/304 (50%)

Seq. ID. No. 236 [TM1;22-44(S), TM2;62-84(S), TM3;97-119(P), TM4;143-165(P), TM5;207-229(P), TM6;242-263(S), TM7;278-300(S)]

olfactory receptor C6 [Mus musculus ] (AF102523);149/305 (48%)

Seq. ID. No. 238 [TM1;34-56(P), TM2;95-117(S), TM3;140-162(P), TM4;166-188(S), TM5;202-224(P), TM6;238-260(S)]

odorant receptor [Mus musculus ] (X92969);201/276 (72%)

Seq. ID. No. 240 [TM1;26-48(P), TM2;69-91(P), TM3;95-117(S), TM4;136-158(P), TM5;166-188(S), TM6;205-227(P), TM7;237-259(S), TM8;271-292(S)]

odorant receptor [Mus musculus ] (X92969);270/309 (87%)

Seq. ID. No. 242 [TM1;26-48(P), TM2;60-82(S), TM3;93-115(P), TM4;144-166(S), TM5;176-198(S), TM6;202-223(P), TM7;234-255(S), TM8;269-291(S)]

odorant receptor MOR18 [Mus musculus ] (AB030895);183/303 (60%)

Seq. ID. No. 244 [TM1;8-30(S), TM2;43-65(S), TM3;87-109(P), TM4;131-153(S), TM5;173-195(S), TM6;233-255(P), TM7;271-292(P), TM8;302-323(P)]

odorant receptor MOR18 [Mus musculus ] (AB030895);195/301 (64%)

Seq. ID. No. 246 [TM1;45-67(P), TM2;112-134(S), TM3;143-165(S), TM4;167-189(S), TM5;192-214(S), TM6;219-240(P), TM7;251-273(P), TM8;281-303(P)]

odorant receptor A16 [Mus musculus ] (AB030896);161/300 (53%)

Seq. ID. No. 248 [TM1;118-140(P), TM2;159-181(P), TM3;186-208(S), TM4;222-244(P), TM5;263-285(P), TM6;294-316(S)]

odorant receptor MOR18 [Mus musculus ] (AB030895);114/193 (59%)

Seq. ID. No. 250 [TM1;2-24(S), TM2;28-49(S), TM3;56-78(S), TM4;91-113(P), TM5;123-145(S), TM6;161-183(S), TM7;201-222(P)]

olfactory receptor OR18—rat(S29710);175/263 (66%)

Seq. ID. No. 252 [TM1;5-27(S), TM2;51-73(P), TM3;123-145(P), TM4;167-189(P), TM5;216-238(P), TM6;261-283(P), TM7;295-317(S)]

odorant receptor MOR18 [Mus musculus ] (AB030895);257/301 (85%)

Seq. ID. No.254 [TM1;28-49(S), TM2;143-165(S), TM3;176-198(S), TM4;202-223(P), TM5;235-257(P), TM6;264-286(S)]

odorant receptor MOR18 [Mus musculus ] (AB030895);169/307 (55%)

Seq. ID. No.256 [TM1;13-35(S), TM2;156-178(P), TM3;192-214(P), TM4;252-274(P), TM5;287-309(P), TM6;313-335(S)]

olfactory receptor OR18-rat(S29710);181/300 (60%)

Seq. ID. No. 258 [TM1;25-47(P), TM2;58-80(P), TM3;93-115(S), TM4;140-162(P), TM5;189-211(P), TM6;237-259(P)]

olfactory receptor OR18—rat(S29710);183/302 (60%)

Seq. ID. No. 260 [TM1;9-31(P), TM2;67-88(S), TM3;90-112(P), TM4;116-138(S), TM5;144-166(P), TM6;179-201(S), TM7;210-231(P), TM8;270-292(S)]

HsOLF1 [Homo sapiens] (U56420);166/307 (54%)

Seq. ID. No. 262 [TM1;20-42(P), TM2;60-82(S), TM3;100-122(S), TM4;146-168(P), TM5;202-224(P), TM6;242-264(S), TM7;270-292(P)]

olfactory receptor [Homo sapiens] (AF065860);159/214 (74%)

Seq. ID. No. 264 [TM1;17-39(S), TM2;91-113(S), TM3;117-139(P), TM4;144-166(P), TM5;183-205(S), TM6;208-229(P), TM7;244-265(S), TM8;274-295(S)]

HsOLF1 [Homo sapiens] (U56420);161/306 (52%)

Seq. ID. No. 266 [TM1;31-53(P), TM2;63-85(S), TM3;102-124(P), TM4;142-164(P), TM5;203-225(P), TM6;241-262(S), TM7;271-293(S)]

HsOLF1 [Homo sapiens] (U56420);180/313 (57%)

Seq. ID. No. 268 [TM1;44-66(P), TM2;70-92(S), TM3;94-116(P), TM4;152-174(P), TM5;182-204(S), TM6;210-232(P)]

olfactory receptor [Homo sapiens] (AF065860);142/216 (65%)

Seq. ID. No. 270 [TM1;45-67(P), TM2;78-100(S), TM3;116-138(P), TM4;158-180(P), TM5;193-215(P), TM6;224-245(P), TM7;258-280(S), TM8;290-311(S)]

olfactory receptor H7 [Mus musculus ] (AF102540);147/222 (66%)

Seq. ID. No. 272 [TM1;29-51(P), TM2;64-86(P), TM3;98-120(P), TM4;143-165(P), TM5;201-223(P), TM6;238-259(S), TM7;269-291(S)]

olfactory receptor C6 [Mus musculus ] (AF102523);140/302 (46%)

Seq. ID. No. 274 [TM1;26-48(P), TM2;61-83(P), TM3;98-120(P), TM4;135-157(P), TM5;196-218(P), TM6;239-260(S)]

olfactory protein [Rattus norvegicus] (M64378);115/258 (44%)

Seq. ID. No. 276 [TM1;48-70(P), TM2;91-113(S), TM3;130-152(S), TM4;176-198(S), TM5;237-259(P), TM6;299-321(S)]

HsOLF1 [Homo sapiens] (U56420);167/303 (55%)

Seq. ID. No. 278 [TM1;32-54(P), TM2;96-118(P), TM3;145-167(P), TM4;201-223(P), TM5;239-261(P), TM6;269-291(S)]

olfactory receptor OR93Gib [Hylobates lar] (AF045580); 166/304 (54%)

Seq. ID. No. 280 [TM1;7-29(P), TM2;35-57(S), TM3;70-92(P), TM4;115-137(S), TM5;177-199(P), TM6;247-268(P)]

olfactory receptor 4 [Gallus gallus ] (X94744);156/282 (55%)

Seq. ID. No.282 [TM1;38-60(P), TM2;103-125(P), TM3;132-154(P), TM4;167-189(P), TM5;207-229(P), TM6;246-267(P), TM7;278-299(P)]

olfactory receptor OR93Gib [Hylobates lar] (AF045580);161/302 (53%)

Seq. ID. No. 284 [TM1;28-50(P), TM2;98-120(P), TM3;140-162(P), TM4;210-232(P), TM5;240-262(S), TM6;272-294(S)]

olfactory receptor OR93Ch [Pan troglodytes] (AF045577);165/308 (53%)

Seq. ID. No. 286 [TM1;1-23(S), TM2;25-47(P), TM3;56-78(S), TM4;90-112(S), TM5;142-164(S), TM6;198-220(P), TM7;236-258(P), TM8;269-290(S)]

olfactory receptor P2 [Mus musculus ] (AF247657);134/302 (44%)

Seq. ID. No. 288 [TM1;93-115(S), TM2;128-150(S), TM3;161-183(P), TM4;212-234(P), TM5;264-286(P), TM6;305-327(S)]

olfactory receptor [Homo sapiens] (AJ003147);183/305 (60%)

Seq. ID. No. 290 [TM1;5-27(S), TM2;40-62(S), TM3;83-105(P), TM4;135-157(P), TM5;181-203(S), TM6;213-235(S)]

olfactory receptor [Homo sapiens] (Z98744);160/250 (64%)

Seq. ID. No. 292 [TM1;30-52(P), TM2;99-121(P), TM3;147-169(P), TM4;207-229(P), TM5;243-265(S), TM6;274-296(S)]

chick olfactory receptor 7 [Gallus gallus ] (Z79586);151/306 (49%)

Seq. ID. No. 294 [TM1;28-50(P), TM2;66-88(S), TM3;104-126(S), TM4;138-160(P), TM5;201-223(P), TM6;242-264(P), TM7;270-292(S)]

olfactory receptor 4 [Gallus gallus ] (X94744);161/310 (51%)

Seq. ID. No. 296 [TM1;20-42(P), TM2;61-83(P), TM3;95-117(S), TM4;143-165(S), TM5;177-199(S), TM6;206-227(P)]

olfactory receptor 2 [Gallus gallus ] (X94742);106/233 (45%)

Seq. ID. No. 298 [TM1;26-48(P), TM2;62-84(S), TM3;104-126(P), TM4;142-164(P), TM5;207-228(P), TM6;271-293(S), TM7;306-328(P)]

odorant receptor [Mus musculus ] (X92969);145/302 (48%)

Seq. ID. No. 300 [TM1;7-29(S), TM2;53-75(P), TM3;101-123(S), TM4;132-154(S), TM5;163-185(P), TM6;198-220(S)]

dJ88J8.1 (novel 7 transmembrane receptor (rhodopsin family) (olfactory receptor like) protein) (hs6M1-15)) [Homo sapiens] (AL035402);173/261 (66%)

Seq. ID. No. 302 [TM1;19-41(S), TM2;83-105(S), TM3;164-186(S), TM4;203-225(P), TM5;239-260(S), TM6;272-293(S)]

olfactory receptor [Homo sapiens] (Z98744);253/310 (81%)

Seq. ID. No. 304 [TM1;8-30(S), TM2;51-73(S), TM3;94-116(P), TM4;172-194(P), TM5;201-223(S), TM6;232-254(S)]

olfactory receptor 4 [Gallus gallus ] (X94744);126/267 (47%)

Seq. ID. No. 306 [TM1;32-54(P), TM2;62-84(S), TM3;134-156(P), TM4;206-228(P), TM5;245-267(S), TM6;276-298(S)]

chick olfactory receptor 4 [Gallus gallus ] (Z79593);154/309 (49%)

Seq. ID. No. 308 [TM1;41-63(P), TM2;73-95(S), TM3;106-128(S), TM4;147-169(P), TM5;219-241(P), TM6;287-309(S)]

HsOLF1 [Homo sapiens] (U56420);146/310 (47%)

Seq. ID. No. 310 [TM1;11-33(S), TM2;42-64(P), TM3;75-97(S), TM4;106-128(S), TM5;151-173(S), TM6;188-210(S), TM7;214-236(P), TM8;249-270(S)]

olfactory receptor P2 [Mus musculus ] (AF247657);276/315 (87%)

Seq. ID. No. 312 [TM1;15-37(S), TM2;53-75(S), TM3;84-106(S), TM4;109-131(P), TM5;161-183(P), TM6;196-218(P)]

olfactory receptor [Homo sapiens]-(AF065870);246/284 (86%)

Seq. ID. No. 314 [TM1;26-48(P), TM2;60-82(S), TM3;99-120(P), TM4;142-164(S), TM5;174-195(S), TM6;202-224(P), TM7;237-258(P), TM8;271-292(S)]

dJ80119.1 (olfactory receptor-like protein (hs6Ml-1)) [Homo sapiens] (AL022727);136/305 (44%)

Seq. ID. No. 316 [TM1;10-32(S), TM2;46-68(P), TM3;80-102(P), TM4;117-139(P), TM5;157-179(P), TM6;222-244(P), TM7;259-281(P)]

odorant receptor S19 [Mus musculus ] (AF121976);168/291 (57%)

Seq. ID. No. 318 [TM1;2-24(S), TM2;45-67(P), TM3;72-94(P), TM4;119-141(S), TM5;170-192(P), TM6;221-243(P), TM7;260-282(P), TM8;288-310(S)]

odorant receptor S19 [Mus musculus ] (AF121976);163/280 (58%)

Seq. ID. No. 320 (TM1;36-58(P), TM2;65-86(S), TM3;93-115(P), TM4;134-156(S), TM5;195-217(P), TM6;236-257(S)]

olfactory receptor-like protein [Homo sapiens] (L35475);280/316 (88%)

Seq. ID. No. 322 [TM1;28-50(P), TM2;96-118(P), TM3;141-163(S), TM4;176-198(S), TM5;207-229(P), TM6;239-260(S), TM7;273-294(S)]

olfactory receptor G3 [Mus musculus ] (AF102535);187/223 (83%)

Seq. ID. No. 324 [TM1;4-26(P), TM2;54-76(S), TM3;84-106(P), TM4;144-166(P), TM5;189-211(S), TM6;214-235(S)]

odorant receptor S25 [Mus musculus ] (AF121977);180/251 (71%)

Seq. ID. No. 326 [TM1;34-56(P), TM2;97-119(P), TM3;140-162(P), TM4;173-195(S), TM5;202-223(P), TM6;266-288(S)]

odorant receptor S25 [Mus musculus ] (AF121977);189/310 (60%)

Seq. ID. No. 328 [TM1;33-55(P), TM2;98-120(P), TM3;142-164(S), TM4;204-226(P), TM5;239-261(S), TM6;271-293(S)]

olfactory receptor P2 [Mus musculus ] (AF247657);158/309 (51%)

Seq. ID. No. 330 [TM1;19-41(P), TM2;61-83(P), TM3;95-117(P), TM4;141-163(P), TM5;201-223(P), TM6;239-260(S), TM7;272-293(S)]

odorant receptor [Mus musculus ] (X92969);156/310 (50%)

Seq. ID. No. 332 [TM1;1-23(S), TM2;33-55(P), TM3;83-105(S), TM4;118-139(P), TM5;166-188(P), TM6;226-248(P), TM7;260-282(S), TM8;292-313(S)]

HGMP07J [Homo sapiens]>gi|228481|prf∥1804351C olfactory receptor HGMP07J [Homo sapiens] (X64995);168/308 (54%)

Seq. ID. No.334 [TM1;33-55(P),TM2;104-126(P),TM3;145-167(S), TM4;207-229(P), TM5;243-265(P), TM6;276-298(P)]

olfactory receptor I7 [Mus musculus ] (AF106007);181/311 (58%)

Seq. ID. No. 336 [TM1;20-42(P), TM2;63-85(P), TM3;96-118(P), TM4;145-167(S), TM5;203-225(P), TM6;238-259(S), TM7;272-293(S)]

HGMP07J [Homo sapiens]>gi|228481|prf∥1804351C olfactory receptor HGMP07J [Homo sapiens] (X64995);160/300 (53%)

Seq. ID. No. 338 [TM1;25-47(P), TM2;61-83(S), TM3;100-122(P), TM4;144-166(P), TM5;198-220(P), TM6;236-258(S), TM7;269-291(S)]

odorant receptor MOR18 [Mus musculus] (AB030895);181/302 (59%)

Seq. ID. No. 340 [TM1;1-23(S), TM2;25-47(P), TM3;59-81(P), TM4;92-114(S), TM5;143-165(S), TM6;204-226(P), TM7;237-259(S), TM8;267-289(S)]

odorant receptor A16 [Mus musculus ] (AB030896);170/297 (57%)

Seq. ID. No. 342 [TM1;23-45(P), TM2;85-107(S), TM3;136-158(P), TM4;171-193(P), TM5;196-217(P), TM6;230-252(P), TM7;259-281(S)]

odorant receptorMOR18 [Mus musculus] (AB030895);184/297 (61%)

Seq. ID. No. 344 [TM1;4-26(P), TM2;29-51(P), TM3;87-109(P), TM4;132-154(P), TM5;200-222(P), TM6;229-250(S)]

olfactory receptor [Mus musculus ] (AJ251154);188/308 (61%)

Seq. ID. No. 346 [TM1;10-32(S), TM2;42-64(P), TM3;76-98(S), TM4;115-137(S), TM5;156-177(S), TM6;216-238(S), TM7;254-276(S), TM8;288-309(S)]

olfactory receptor-like protein COR3′beta [Gallus gallus ] (L17432);167/314 (53%)

Seq. ID. No. 348 [TM1;2-23(S), TM2;29-51(S), TM3;67-89(S), TM4;102-124(P), TM5;161-183(S), TM6;209-231(S), TM7;237-259(S)]

olfactory receptor [Homo sapiens] (Y10529);193/270 (71%)

Seq. ID. No. 350 [TM1;38-60(P), TM2;81-103(S), TM3;111-133(P), TM4;154-176(S), TM5;218-240(P), TM6;254-276(P), TM7;288-309(S)]

olfactory receptor [Mus musculus domesticus] (AF073987);183/216 (84%)

Seq. ID. No. 352 [TM1;1-23(S), TM2;30-52(P), TM3;64-86(P), TM4;103-125(S), TM5;144-165(P), TM6;208-230(P), TM7;242-264(S), TM8;276-297(S)]

odorant receptor S18 [Mus musculus ] (AF121975);219/305 (71%)

Seq. ID. No. 354 [TM1;38-60(P), TM2;71-93(P), TM3;107-129(S), TM4;152-174(S), TM5;213-235(P), TM6;248-270(S), TM7;280-302(S)]

odorant receptor S19 [Mus musculus ] (AF121976);163/288 (56%)

Seq. ID. No. 356 [TM1;28-50(P), TM2;72-94(S), TM3;112-134(P), TM4;141-163(S), TM5;221-243(P), TM6;286-308(P), TM7;326-347(S), TM8;357-379(S)]

olfactory receptor C6 [Mus musculus ] (AF102523);138/308 (44%)

Seq. ID. No. 358 [TM1;29-51(P), TM2;66-88(P), TM3;106-128(S), TM4;132-154(P), TM5;197-219(P), TM6;234-255(S), TM7;267-289(S)]

olfactory receptor C6 [Mus musculus ] (AF102523);140/310 (45%)

Seq. ID. No. 360 [TM1;1-23(S), TM2;28-50(S), TM3;64-86(P), TM4;100-122(P), TM5;166-188(P), TM6;201-222(S)]

olfactory receptor C6 [Mus musculus ] (AF102523);100/215 (46%)

Seq. ID. No. 362 [TM1;32-54(P),TM2;105-127(P),TM3;144-166(P), TM4;185-207(S), TM5;210-231(P), TM6;241-263(S), TM7;272-294(S)]

HsOLF1 [Homo sapiens] (U56420);161/310 (51%)

Seq. ID. No. 364 [TM1;19-41(P), TM2;61-83(S), TM3;90-112(P), TM4;116-138(S), TM5;145-167(S), TM6;179-201(S), TM7;210-232(P), TM8;270-292(S)]

HsOLF1 [Homo sapiens] (U56420);168/307 (54%)

Seq. ID. No. 366 [TM1;19-41(S), TM2;60-82(S), TM3;99-121(S), TM4;140-162(P), TM5;206-227(P), TM6;243-264(S), TM7;273-293(S)]

CfOLF2 [Canis familiaris] (U53680);175/308 (56%)

Seq. ID. No.368 [TM1;29-51(P),TM2;101-123(P),TM3;144-166(S), TM4;206-227(P), TM5;240-262(S), TM6;273-295(S)]

olfactory receptor OR93Ch [Pan troglodytes] (AF045577);162/313 (51%)

Seq. ID. No. 370 [TM1;23-45(P), TM2;66-87(S), TM3;94-116(S), TM4;133-155(S), TM5;200-222(P), TM6;246-268(S), TM7;276-298(S)]

CfOLF1 [Canis familiaris] (U53679);153/299 (51%)

Seq. ID. No. 372 [TM1;41-63(P),TM2;102-124(S),TM3;137-159(P), TM4;207-229(P), TM5;246-268(P), TM6;278-300(P)]

HsOLF1 [Homo sapiens] (U56420);173/306 (56%)

Seq. ID. No. 374 [TM1;7-29(S), TM2;32-53(P), TM3;60-82(S), TM4;100-122(S), TM5;144-166(P), TM6;200-222(P), TM7;235-257(S)]

similar to rat olfactory receptor OR18; similar to S29710 (PID:g423702) [Homo sapiens] (AC004908);220/311 (70%)

Seq. ID. No. 376 [TM1;1-23(S), TM2;28-50(P), TM3;59-81(S), TM4;95-117(P), TM5;142-164(P), TM6;206-228(P), TM7;239-260(P)]

similar to rat olfactory receptor OR18; similar to S29710 (PID:g423702) [Homo sapiens] (AC004908);225/291 (77%)

Seq. ID. No.378 [TM1;9-31(S), TM2;123-145(P), TM3;148-170(S), TM4;192-214(S), TM5;236-258(P), TM6;295-317(P), TM7;334-355(P), TM8;367-389(S)]

olfactory receptor OR18—rat(S29710);204/302 (67%)

Seq. ID. No. 380 [TM1;9-31(S), TM2;34-56(P), TM3;62-84(S), TM4;97-119(S), TM5;144-166(S), TM6;205-227(P), TM7;242-263(P), TM8;275-296(P)]

olfactory receptor 4 [Gallus gallus ] (X94744);173/303 (57%)

Seq. ID. No. 382 [TM1;20-42(S), TM2;62-84(S), TM3;95-117(S), TM4;146-168(S), TM5;205-226(P), TM6;237-259(S), TM7;268-290(S)]

odorant receptor MOR83 [Mus musculus ] (AB030894);156/304 (51%)

Seq. ID. No. 384 [TM1;22-44(P), TM2;62-84(S), TM3;98-120(S), TM4;144-166(P), TM5;197-219(P), TM6;237-259(S), TM7;269-291(S)]

odorant receptor MOR83 [Mus musculus ] (AB030894);159/304 (52%)

Seq. ID. No. 386 [TM1;49-71(P), TM2;77-99(S), TM3;120-142(P), TM4;173-195(S), TM5;221-243(P), TM6;260-282(P), TM7;289-311(P)]

MOR 3′Betal [Mus musculus ] (AF133300);188/310 (60%)

Seq. ID. No. 388 [TM1;17-39(P), TM2;50-72(S), TM3;82-104(P), TM4;134-156(S), TM5;167-189(S), TM6;196-217(P), TM7;224-245(S)]

olfactory receptor P2 [Mus musculus ] (AF247657);226/296 (76%)

Seq. ID. No. 390 [TM1;19-41(P), TM2;61-83(P), TM3;94-116(P), TM4;136-158(P), TM5;178-200(S), TM6;207-229(P), TM7;273-295(S)]

olfactory receptor P2 [Mus musculus ] (AF247657);170/304 (55%)

Seq. ID. No. 392 [TM1;27-49(P), TM2;62-84(P), TM3;95-117(P), TM4;135-157(P), TM5;177-199(S), TM6;207-229(P), TM7;273-295(S)]

olfactory receptor P2 [Mus musculus ] (AF247657);164/304 (53%)

Seq. ID. No. 394 [TM1;30-52(P), TM2;74-96(P), TM3;137-157(P), TM4;183-205(S), TM5;258-280(P), TM6;320-341(S)]

taste bud receptor protein TB 567 [Rattus norvegicus] (U50948);235/307 (76%)

Seq. ID. No. 396 [TM1;24-46(P), TM2;56-78(S), TM3;94-116(S), TM4;129-151(P), TM5;207-229(P), TM6;241-263(S), TM7;272-292(P)]

olfactory receptor OR14-rat(S29709);256/304 (84%)

Seq. ID. No. 398 [TM1;28-50(P), TM2;98-120(P), TM3;127-149(P), TM4;203-225(P), TM5;249-271(S), TM6;274-295(S)]

olfactory receptor OR14-rat(S29709);234/299 (78%)

Seq. ID. No. 400 [TM1;32-54(P), TM2;57-79(P), TM3;92-114(P), TM4;129-151(P), TM5;201-223(P), TM6;237-259(S)]

olfactory receptor OR14-rat(S29709);189/302 (62%)

Seq. ID. No. 402 [TM1;30-52(P), TM2;62-84(S), TM3;94-116(P), TM4;138-159(P), TM5;203-225(P), TM6;238-259(S), TM7;272-293(S)]

olfactory receptor [Rattus norvegicus] (X80671);210/305 (68%)

Seq. ID. No. 404 [TM1;26-48(P), TM2;94-116(S), TM3;129-151(S), TM4;181-203(P), TM5;208-229(P), TM6;240-262(S), TM7;271-292(S)]

olfactory receptor OR14-rat(S29709);201/300 (67%)

Seq. ID. No. 406 [TM1;28-50(P), TM2;63-85(S), TM3;93-115(P), TM4;130-152(P), TM5;202-224(P), TM6;238-259(P)]

olfactory receptor [Rattus norvegicus] (X80671);247/302 (81%)

Seq. ID. No. 408 [TM1;1-21(S), TM2;40-62(P), TM3;98-120(P), TM4;223-245(P), TM5;260-282(S), TM6;290-312(S)]

taste bud receptor protein TB 641 [Rattus norvegicus](U50949);185/297 (62%)

Seq. ID. No.410 [TM1;20-42(P), TM2;200-222(P), TM3;239-261(P), TM4;269-290(S), TM5;304-326(P), TM6;331-352(P), TM7;358-372(S)]

taste bud receptor protein TB 641 [Rattus norvegicus] (U50949);170/302 (56%)

Seq. ID. No. 412 [TM1;1-23(S), TM2;26-48(S), TM3;110-132(S), TM4;137-158(P), TM5;170-192(P), TM6;198-220(S)]

taste bud receptor protein TB 641 [Rattus norvegicus] (U50949);140/234 (59%)

Seq. ID. No. 414 [TM1;3-25(S), TM2;30-52(P), TM3;63-85(S), TM4;180-202(S), TM5;205-226(P), TM6;239-261(P), TM7;266-288(S)]

taste bud receptor protein TB 641 [Rattus norvegicus] (U50949);165/299 (55%)

Seq. ID. No. 416 [TM1;6-28(S), TM2;41-63(S), TM3;102-124(P), TM4;142-164(P), TM5;170-192(S), TM6;198-220(S)]

olfactory receptor OR18-rat(S29710);154/209 (73%)

Seq. ID. No. 418 [TM1;26-48(S), TM2;99-121(P), TM3;141-163(P), TM4;204-226(P), TM5;235-256(P), TM6;267-289(S), TM7;305-327(P)]

odorant receptor A16 [Mus musculus ] (AB030896);164/286 (57%)

Seq. ID. No. 420 [TM1;33-55(P), TM2;70-92(S), TM3;103-125(P), TM4;209-231(P), TM5;247-269(S), TM6;278-300(S)]

olfactory receptor 4 [Gallus gallus ] (X94744);159/306 (51%)

Seq. ID. No. 422 [TM1;6-28(S), TM2;31-52(P), TM3;91-113(P), TM4;137-159(S), TM5;201-223(P), TM6;239-261(S), TM7;270-292(S)]

olfactory receptor OR93Ch [Pan troglodytes] (AF045577);166/305 (54%)

Seq. ID. No. 424 [TM1;24-46(P), TM2;99-120(P), TM3;137-159(P), TM4;204-225(P), TM5;237-258(S), TM6;270-291(S)]

HsOLF1 [Homo sapiens] (U56420);154/301 (51%)

Seq. ID. No. 426 [TM1;17-39(S), TM2;72-94(S), TM3;134-156(P), TM4;203-224(P), TM5;238-260(P), TM6;269-291(S)]

olfactory receptor 2 [Gallus gallus ] (X94742);155/307 (50%)

Seq. ID. No. 428 [TM1;26-48(P), TM2;55-77(S), TM3;81-103(S), TM4;141-163(P), TM5;197-219(P), TM6;238-260(P), TM7;268-290(S)]

olfactory receptor OR18—rat(S29710);193/299 (64%)

Seq. ID. No. 430 [TM1;35-57(S), TM2;96-118(S), TM3;130-152(P), TM4;156-178(S), TM5;193-215(P), TM6;239-261(P), TM7;266-288(S), TM8;294-315(S)]

olfactory receptor OR18-rat(S29710);213/304 (70%)

Seq. ID. No. 432 [TM1;10-32(S), TM2;63-85(P), TM3;101-123(S), TM4;158-180(P), TM5;194-216(P), TM6;229-249(S)]

olfactory receptor [Rattus norvegicus] (AF010293);198/263 (75%)

Seq. ID. No. 434 [TM1;16-38(P), TM2;56-78(S), TM3;86-108(P), TM4;139-161(P), TM5;197-219(P), TM6;238-260(S), TM7;270-291(S)]

olfactory protein [Rattus norvegicus] (M64378);142/294 (48%)

Seq. ID. No. 436 [TM1;8-30(P), TM2;60-82(S), TM3;93-115(P), TM4;139-161(P), TM5;203-225(P), TM6;268-290(S)]

olfactory receptor C6 [Mus musculus ] (AF102523);149/312 (47%)

Seq. ID. No. 438 [TM1;21-43(P), TM2;89-111(S), TM3;132-154(S), TM4;199-221(P), TM5;239-260(S), TM6;269-291(S)]

olfactory receptor 2 [Gallus gallus ] (X94742);148/304 (48%)

Seq. ID. No. 440 [TM1;34-56(P), TM2;62-84(P), TM3;88-110(S), TM4;206-228(P), TM5;238-260(P), TM6;270-291(S)]

olfactory receptor 2 [Gallus gallus ] (X94742);158/307 (51%)

Seq. ID. No. 442 [TM1;23-45(S), TM2;76-98(S), TM3;134-156(S), TM4;199-221(P), TM5;240-261(S), TM6;270-291(P)]

olfactory receptor 4 [Gallus gallus ] (X94744);171/301 (56%)

Seq. ID. No. 444 [TM1;3-25(S), TM2;37-59(S), TM3;78-100(P), TM4;147-169(P), TM5;184-206(P), TM6;214-235(S)]

olfactory receptor OR93Ch [Pan troglodytes] (AF045577);134/245 (54%)

Seq. ID. No. 446 [TM1;8-30(P), TM2;33-54(P), TM3;95-117(S), TM4;126-148(P), TM5;208-230(P), TM6;271-293(S)]

olfactory receptor 4 [Gallus gallus ] (X94744);149/304 (49%)

Seq. ID. No. 448 [TM1;57-79(P), TM2;96-118(S), TM3;131-153(S), TM4;205-227(S), TM5;237-259(P), TM6;274-295(S)]

taste bud receptor protein TB 641 [Rattus norvegicus] (U50949);173/298 (58%)

Seq. ID. No. 450 [TM1;19-41(P), TM2;62-84(P), TM3;95-117(P), TM4;141-163(P), TM5;202-224(P), TM6;240-261(P), TM7;272-294(S)]

olfactory receptor C6 [Mus musculus ] (AF102523);152/300 (50%)

Seq. ID. No. 452 [TM1;31-53(P), TM2;95-117(P), TM3;125-147(P), TM4;180-202(S), TM5;208-229(P), TM6;238-260(S), TM7;272-294(P)]

olfactory receptor [Rattus norvegicus] (X80671);193/301 (64%)

Seq. ID. No. 454 [TM1;2-24(S), TM2;29-51(S), TM3;59-81(S), TM4;107-129(S), TM5;169-190(P), TM6;201-223(P)]

odorant receptor MOR83 [Mus musculus] (AB030894);156/269 (57%)

Seq. ID. No. 456 [TM1;1-21(S), TM2;40-62(P), TM3;98-120(P), TM4;223-245(P), TM5;259-281(S), TM6;298-320(P)]

taste bud receptor protein TB 641 [Rattus norvegicus] (U50949);154/249 (61%)

Seq. ID. No. 458 [TM1;25-47(P), TM2;55-77(S), TM3;97-119(S), TM4;140-162(S), TM5;200-222(P), TM6;236-258(S)]

olfactory receptor 4 [Gallus gallus ] (X94744);153/303 (50%)

Seq. ID. No. 460 [TM1;1-17(S), TM2;27-49(S), TM3;67-89(P), TM4;102-124(P), TM5;174-196(S), TM6;208-230(P), TM7;238-260(S)]

olfactory receptor [Homo sapiens] (Y10529);185/261 (70%)

Seq. ID. No. 462 [TM1;1-20(S), TM2;57-79(P), TM3;89-111(S), TM4;122-144(P), TM5;156-178(P), TM6;224-246(P)]

olfactory receptor 4 [Gallus gallus ] (X94744);122/229 (53%)

Seq. ID. No. 464 [TM1;32-54(S), TM2;92-114(P), TM3;140-162(S), TM4;197-219(S), TM5;238-260(S), TM6;272-293(P)]

olfactory receptor OR93Gib [Hylobates lar] (AF045580); 160/309 (51%)

Seq. ID. No. 466 [TM1;1-23(S), TM2;33-55(S), TM3;69-91(P), TM4;150-171(P), TM5;182-204(P), TM6;211-233(S)]

HsOLF1 [Homo sapiens] (U56420);147/247 (59%)

Seq. ID. No. 468 [TM1;26-48(P), TM2;94-116(P), TM3;136-158(P), TM4;180-202(S), TM5;209-231(P), TM6;238-260(S), TM7;271-292(P)]

olfactory receptor 4 [Gallus gallus ] (X94744);170/307 (55%)

Seq. ID. No. 470 [TM1;7-29(P), TM2;35-57(S), TM3;70-92(S), TM4;184-205(P), TM5;213-235(S), TM6;247-268(P)]

olfactory receptor OR93Gib [Hylobates lar] (AF045580);151/283 (53%)

Seq. ID. No. 472 [TM1;19-41(P), TM2;56-78(S), TM3;104-126(P), TM4;144-166(P), TM5;203-224(P), TM6;237-259(P), TM7;269-291(P)]

odorant receptor MOR83 [Mus musculus ] (AB030894);160/306 (52%)

Seq. ID. No.474 [TM1;27-49(P),TM2;142-164(S), TM3;172-193(S), TM4;205-226(P), TM5;242-264(P), TM6;268-290(S)]

odorant receptor MOR83 [Mus musculus ] (AB030894);170/308 (55%)

Seq. ID. No.476 [TM1;37-59(P), TM2;108-130(S), TM3;153-175(P), TM4;188-210(P), TM5;214-235(P), TM6;251-273(P), TM7;278-300(S)]

odorant receptor MOR83 [Mus musculus ] (AB030894);168/297 (56%)

Seq. ID. No. 478 [TM1;29-51(P), TM2;58-80(S), TM3;141-163(P), TM4;177-199(P), TM5;205-226(P), TM6;239-261(P), TM7;271-293(S)]

odorant receptor MOR83 [Mus musculus ] (AB030894);159/300 (53%)

Seq. ID. No.480 [TM1;30-52(P), TM2;95-117(S), TM3;149-171(S), TM4;197-219(P), TM5;241-263(P), TM6;268-290(P)]

similar to rat olfactory receptor OR18; similar to S29710 (PID:g423702) [Homo sapiens] (AC004908);167/312 (53%)

Seq. ID. No. 482 [TM1;23-45(P), TM2;58-80(S), TM3;96-118(S), TM4;141-163(S), TM5;200-222(P), TM6;245-267(S), TM7;270-292(S)]

CfOLF1 [Canis familiaris] (U53679);149/310 (48%)

Seq. ID. No. 484 [TM1;29-50(P), TM2;60-82(S), TM3;95-117(P), TM4;134-156(S), TM5;203-225(P), TM6;238-259(S), TM7;274-294(S)]

olfactory receptor P2 [Mus musculus ] (AF247657);144/307 (46%)

Seq. ID. No.486 [TM1;26-48(S), TM2;88-110(P), TM3;136-158(S), TM4;194-216(P), TM5;231-253(P), TM6;259-281(S)]

similar to rat olfactory receptor OR18; similar to S29710 (PID:g423702) [Homo sapiens] (AC004908);177/296 (59%)

Seq. ID. No. 488 [TM1;30-52(P), TM2;70-92(S), TM3;106-128(S), TM4;146-168(P), TM5;196-218(P), TM6;239-261(S), TM7;270-292(S)]

odorant receptor S46 [Mus musculus ] (AF121979);188/304 (61%)

Seq. ID. No. 490 [TM1;25-47(P), TM2;53-75(S), TM3;150-172(S), TM4;199-221(P), TM5;235-257(P), TM6;269-291(S)]

odorant receptor MOR18 [Mus musculus ] (AB030895);193/301 (64%)

Seq. ID. No. 492 [TM1;6-28(S), TM2;30-51(S), TM3;105-127(P), TM4;138-160(P), TM5;169-191(S), TM6;199-221(P), TM7;235-257(P), TM8;266-288(S)]

odorant receptor A16 [Mus musculus ] (AB030896);184/302 (60%)

Seq. ID. No. 494 [TM1;24-46(P), TM2;57-79(S), TM3;108-130(S), TM4;145-167(S), TM5;177-199(S), TM6;209-231(P), TM7;239-261(S), TM8;270-292(P)]

odorant receptor S1 [Mus musculus ] (AF121972);129/297 (43%)

Seq. ID. No. 496 [TM1;74-96(P),TM2;126-148(S),TM3;169-191(P), TM4;206-228(P), TM5;234-255(P), TM6;266-288(P), TM7;297-319(P)]

olfactory receptor P2 [Mus musculus ] (AF247657);130/305 (42%)

Seq. ID. No. 498 [TM1;48-70(P), TM2;73-95(S), TM3;99-121(S), TM4;130-151(S), TM5;162-184(P), TM6;222-244(P), TM7;254-275(P), TM8;284-306(S)]

chick olfactory receptor 7 [Gallus gallus ] (Z79586);130/303 (42%)

Seq. ID. No. 500 [TM1;8-30(P), TM2;34-56(P), TM3;82-104(S), TM4;140-162(P), TM5;202-224(P), TM6;240-261(P), TM7;269-291(S)]

odorant receptor S1 [Mus musculus ] (AF121972);143/307 (46%)

Seq. ID. No. 502 [TM1;1-23(S), TM2;42-64(S), TM3;84-106(P), TM4;148-169(P), TM5;184-205(S), TM6;213-235(S)]

olfactory receptor E6 [Mus musculus ] (AF102528);155/223 (69%)

Seq. ID. No. 504 [TM1;2-24(S), TM2;40-62(S), TM3;80-102(P), TM4;144-166(P), TM5;181-202(S), TM6;214-235(S)]

olfactory receptor [Rattus norvegicus] (X80671);127/243 (52%)

Seq. ID. No. 506 [TM1;1-23(P), TM2;27-49(P), TM3;65-87(S), TM4;89-111(P), TM5;156-178(P), TM6;194-215(P), TM7;227-249(S)]

olfactory receptor [Gorilla gorilla] (AF101764);93/214 (43%)

Seq. ID. No. 508 [TM1;11-33(P), TM2;49-71(S), TM3;80-102(S), TM4;128-150(S), TM5;193-215(P), TM6;225-247(P), TM7;254-276(S)]

olfactory receptor P2 [Mus musculus ] (AF247657);128/290 (44%)

Seq. ID. No. 510 [TM1;18-40(P), TM2;56-78(P), TM3;91-113(S), TM4;125-147(S), TM5;200-222(P), TM6;242-264(S), TM7;269-291(S)]

olfactory receptor-like protein [Rattus norvegicus] (AF029357);129/300 (43%)

Seq. ID. No. 512 [TM1;25-47(P), TM2;55-77(S), TM3;93-115(P), TM4;124-146(S), TM5;206-228(P), TM6;237-259(P), TM7;270-291(S)]

olfactory receptor [Gorilla gorilla] (AF101764);125/302 (41%)

Seq. ID. No. 514 [TM1;8-30(S), TM2;32-51(P), TM3;63-85(S), TM4;94-116(S), TM5;141-163(P), TM6;201-223(P), TM7;244-266(S), TM8;272-293(S)]

olfactory receptor-like protein [Rattus norvegicus] (AF029357);131/297 (44%)

Seq. ID. No. 516 [TM1;26-48(P), TM2;53-75(S), TM3;96-118(S), TM4;135-157(P), TM5;198-220(P), TM6;241-263(S), TM7;270-291(S)]

gustatory receptor 43 [Rattus norvegicus] (AB038167);265/311 (85%)

Seq. ID. No. 518 [TM1;31-52(P), TM2;62-84(S), TM3;99-121(P), TM4;144-166(S), TM5;202-224(P), TM6;236-258(P), TM7;271-292(S)]

dJ80119.7 (olfactory receptor-like protein (hs6M1-3)) [Homo sapiens] (AL022727);174/304 (57%)

Seq. ID. No. 520 [TM1;8-30(P), TM2;79-101(P), TM3;128-150(S), TM4;186-208(P), TM5;226-247(S), TM6;257-277(S)]

gustatory receptor 43 [Rattus norvegicus] (AB038167);169/283 (59%)

Seq. ID. No. 522 [TM1;32-54(P), TM2;56-78(S), TM3;91-113(S), TM4;142-164(S), TM5;205-227(P), TM6;238-260(S), TM7;272-294(S)]

olfactory protein [Rattus norvegicus] (M64377);161/303 (53%)

Seq. ID. No. 524 [TM1;1-22(S), TM2;29-51(S), TM3;101-123(P), TM4;172-194(P), TM5;213-235(S), TM6;239-261(S)]

olfactory protein [Rattus norvegicus] (M64392);165/268 (61%)

Seq. ID. No. 526 [TM1;28-50(P), TM2;61-83(S), TM3;103-125(P), TM4;140-162(P), TM5;202-224(P), TM6;237-259(P)]

olfactory protein [Rattus norvegicus] (M64377);161/305 (52%)

Seq. ID. No. 528 [TM1;27-49(P), TM2;63-85(S), TM3;91-113(S), TM4;141-163(S), TM5;177-199(S), TM6;206-228(P), TM7;240-262(S), TM8;270-292(S)]

olfactory receptor [Pan troglodytes] (AF101741);195/307 (63%)

Seq. ID. No. 530 [TM1;2-24(S), TM2;31-53(S), TM3;65-87(S), TM4;109-131(S), TM5;169-191(P), TM6;209-226(S)]

olfactory receptor [Mus musculus ] (AJ251154);190/285 (66%)

Seq. ID. No.532 [TM1;33-55(P), TM2;98-120(S), TM3;132-154(S), TM4;175-197(S), TM5;205-227(P), TM6;237-258(P), TM7;278-299(S)]

olfactory factor OR37-rat(S29711); 244/304 (80%)

Seq. ID. No. 534 [TM1;2-24(S), TM2;31-53(S), TM3;65-87(S), TM4;109-131(S), TM5;171-193(P), TM6;208-226(P)]

olfactory receptor [Mus musculus ] (AJ251154);197/285 (69%)

Seq. ID. No. 536 [TM1;1-23(S), TM2;57-79(P), TM3;128-150(S), TM4;169-191(S), TM5;206-228(P), TM6;237-259(P), TM7;267-288(P), TM8;305-327(S)]

olfactory factor OR37—rat(S29711);259/304 (85%)

Seq. ID. No. 538 [TM1;8-30(P), TM2;56-78(S), TM3;81-103(S), TM4;107-129(S), TM5;148-170(P), TM6;177-199(P), TM7;203-225(P)]

olfactory receptor [Mus musculus ] (AJ251154);166/283 (58%)

Seq. ID. No. 540 [TM1;24-46(P), TM2;62-84(S), TM3;101-123(S), TM4;137-159(S), TM5;171-193(S), TM6;207-229(P), TM7;237-258(P), TM8;274-296(S)]

olfactory receptor [Mus musculus ] (AJ251154);219/319 (68%)

Seq. ID. No. 542 [TM1;43-65(P),TM2;117-139(S),TM3;152-174(P), TM4;214-236(P), TM5;253-274(S), TM6;286-306(S)]

olfactory protein [Rattus norvegicus] (M64377);174/311 (55%)

Seq. ID. No. 544 [TM1;39-61(P),TM2;116-138(P),TM3;152-174(P), TM4;214-236(P), TM5;253-274(P), TM6;284-306(S)]

olfactory receptor [Homo sapiens] (AJ003147);171/308 (55%)

Seq. ID. No. 546 [TM1;33-55(P), TM2;68-90(P), TM3;100-122(S), TM4;143-165(S), TM5;208-230(P), TM6;244-266(S), TM7;279-298(S)]

HGMP07J [Homo sapiens]>gi|228481|prf∥1804351C olfactory receptor HGMP07J [Homo sapiens] (X64995);143/297 (48%)

Seq. ID. No. 548 [TM1;17-39(P), TM2;70-92(S), TM3;98-120(P), TM4;145-167(P), TM5;202-224(P), TM6;238-259(S)]

olfactory receptor OR93Gib [Hylobates lar] (AF045580); 149/309 (48%)

Seq. ID. No. 550 [TM1;1-19(S), TM2;27-49(P), TM3;103-125(S), TM4;139-161(P), TM5;201-223(P), TM6;237-258(S)]

olfactory factor OR37-rat(S29711);148/305 (48%)

Seq. ID. No. 552 [TM1;33-54(P), TM2;60-82(P), TM3;95-117(P), TM4;133-155(S), TM5;202-224(P), TM6;239-261(P), TM7;273-294(S)]

olfactory receptor E3 [Mus musculus ] (AF102527);159/223 (71%)

Seq. ID. No. 554 [TM1;19-41(S), TM2;49-71(P), TM3;81-103(S), TM4;121-143(P), TM5;163-185(S), TM6;216-238(P), TM7;260-282(S), TM8;294-314(S)]

olfactory receptor E3 [Mus musculus ] (AF102527);168/223 (75%)

Seq. ID. No. 556 [TM1;33-55(P), TM2;65-87(P), TM3;101-123(S), TM4;149-171(P), TM5;208-230(P), TM6;242-264(P), TM7;277-298(S), TM8;320-342(P)]

olfactory receptor E3 [Mus musculus ] (AF102527);128/223 (57%)

Seq. ID. No. 558 [TM1;4-26(S), TM2;34-56(S), TM3;62-84(S), TM4;165-187(P), TM5;204-226(P), TM6;235-257(S)]

olfactory receptor E3 [Mus musculus ] (AF102527);147/223 (65%)

Seq. ID. No. 560 [TM1;25-47(P), TM2;57-79(S), TM3;93-115(P), TM4;134-156(S), TM5;201-223(S), TM6;234-256(S), TM7;269-291(S)]

olfactory receptor [Gorilla gorilla] (AF101764);128/301 (42%)

Seq. ID. No.562 [TM1;34-55(P),TM2;101-123(S),TM3;144-166(S), TM4;208-230(P), TM5;244-266(P), TM6;275-297(S)]

olfactory receptor E3 [Mus musculus ] (AF102527);152/223 (68%)

Seq. ID. No. 564 [TM1;35-57(P), TM2;95-117(S), TM3;142-164(P), TM4;170-192(S), TM5;207-229(P), TM6;238-260(P)]

olfactory receptor [Mus musculus ] (AJ251155);256/312 (82%)

Seq. ID. No. 566 [TM1;19-41(P), TM2;61-83(P), TM3;95-117(S), TM4;141-163(S), TM5;201-223(P), TM6;239-260(S)]

HGMP07J [Homo sapiens]>gi|228481∥prf∥1804351C olfactory receptor HGMP07J [Homo sapiens] (X64995);157/309 (50%)

Seq. ID. No. 568 [TM1;24-46(P), TM2;57-79(S), TM3;92-114(S), TM4;141-163(P), TM5;202-224(P), TM6;239-261(S), TM7;268-290(S)]

olfactory protein [Rattus norvegicus] (M64378);208/304 (68%)

Seq. ID. No. 570 [TM1;63-85(P), TM2;98-120(S), TM3;145-167(S), TM4;179-201(S), TM5;243-265(P), TM6;281-302(S)]

olfactory receptor [Mus musculus ] (M84005);191/305 (62%)

Seq. ID. No. 572 [TM1;13-35(S), TM2;59-81(S), TM3;102-124(S), TM4;166-188(P), TM5;201-223(S), TM6;237-257(S)]

OL1 receptor [Rattus norvegicus] (L34074);120/270 (44%)

Seq. ID. No. 574 [TM1;15-37(S), TM2;52-74(S), TM3;99-121(S), TM4;162-184(P), TM5;195-216(S), TM6;227-249(S), TM7;252-274(P), TM8;277-299(P)]

olfactory receptor G7 [Mus musculus ] (AF102537);133/223 (59%)

Seq. ID. No. 576 [TM1;19-41(P), TM2;57-79(S), TM3;94-116(S), TM4;139-161(S), TM5;202-224(P), TM6;240-260(S), TM7;276-298(S)]

olfactory receptor P2 [Mus musculus ] (AF247657);135/301 (44%)

Seq. ID. No. 578 [TM1;28-50(P), TM2;62-84(S), TM3;98-120(P), TM4;144-166(P), TM5;209-231(S), TM6;242-264(P)]

olfactory protein [Rattus norvegicus] (M64377);175/309 (56%)

Seq. ID. No. 580 [TM1;1-23(S), TM2;51-73(P), TM3;119-141(S), TM4;166-187(S), TM5;204-226(S), TM6;235-257(P), TM7;272-294(S)]

CfOLF2 [Canis familiaris] (U53680);168/293 (57%)

Seq. ID. No.582 [TM1;32-54(P), TM2;94-116(S), TM3;145-167(P), TM4;200-222(P), TM5;246-268(S), TM6;270-292(S)]

olfactory receptor [Pan troglodytes] (AF101741);184/307 (59%)

Seq. ID. No. 584 [TM1;55-77(P), TM2;91-113(S), TM3;124-146(P), TM4;172-194(S), TM5;209-231(P), TM6;238-260(P), TM7;265-287(P), TM8;298-320(S)]

olfactory receptor [Mus musculus ] (AJ251155);179/306 (58%)

Seq. ID. No.586 [TM1;27-49(P), TM2;87-109(P), TM3;126-148(S), TM4;153-175(S), TM5;202-224(P), TM6;239-260(S), TM7;272-294(S)]

olfactory receptor [Rattus norvegicus] (X80671);151/300 (50%)

Seq. ID. No. 588 [TM1;28-50(P), TM2;98-120(P), TM3;140-162(P), TM4;206-227(P), TM5;240-262(S), TM6;271-293(S)]

olfactory receptor OR93Ch [Pan troglodytes] (AF045577);161/304 (52%)

Seq. ID. No. 590 [TM1;11-33(S), TM2;52-73(S), TM3;98-120(P), TM4;135-157(S), TM5;163-184(P), TM6;225-247(S)]

HGMP07J [Homo sapiens]>gi|228481|prf∥1804351C olfactory receptor HGMP07J [Homo sapiens] (X64995);119/261 (45%)

Seq. ID. No. 592 [TM1;1-23(P), TM2;42-64(S), TM3;76-98(S), TM4;107-129(P), TM5;137-158(S), TM6;176-198(S)]

olfactory receptor [Mus musculus ] (AJ251154);188/216 (87%)

Seq. ID. No. 594 [TM1;11-33(P), TM2;47-69(S), TM3;94-116(P), TM4;140-162(P), TM5;168-189(S), TM6;195-208(S)]

similar to mouse olfactory receptor 13; similar to P34984 (PID:g464305) [Homo sapiens] (AC005587);126/208 (60%)

Seq. ID. No. 596 [TM1;17-39(P), TM2;84-106(P), TM3;126-147(S), TM4;149-171(S), TM5;201-223(S), TM6;258-280(P), TM7;298-320(S), TM8;330-352(S)]

olfactory receptor [Mus musculus ] (M84005);182/305 (59%)

Seq. ID. No. 598 [TM1;16-38(P), TM2;94-116(S), TM3;140-162(P), TM4;171-193(S), TM5;200-222(P), TM6;239-261(S)]

similar to mouse olfactory receptor 13; similar to P34984 (PID:g464305) [Homo sapiens] (AC005587);183/251 (72%)

Seq. ID. No. 600 [TM1;32-54(P), TM2;95-117(P), TM3;139-161(S), TM4;196-218(P), TM5;242-264(S), TM6;276-298(S)]

HsOLF3 [Homo sapiens] (U56421);135/305 (44%)

Seq. ID. No. 602 [TM1;19-41(P), TM2;61-83(P), TM3;94-116(S), TM4;148-170(P), TM5;180-202(S), TM6;211-233(P), TM7;241-262(S), TM8;273-294(S)]

R30385_{—}1 [Homo sapiens] (AC004510);293/313 (93%)

Seq. ID. No.604 [TM1;73-95(S), TM2;146-168(S), TM3;191-213(S), TM4;245-267(P), TM5;291-312(P), TM6;324-344(S)]

olfactory receptor 4 [Gallus gallus ] (X94744);169/310 (54%)

Seq. ID. No. 606 [TM1;1-21(S), TM2;45-67(P), TM3;73-95(S), TM4;106-128(S), TM5;161-183(P), TM6;223-245(P), TM7;258-280(S), TM8;290-311(S)]

odorant receptor S1 [Mus musculus ] (AF121972);215/315 (68%)

Seq. ID. No. 608 [TM1;41-63(P), TM2;71-93(S), TM3;105-127(P), TM4;149-171(P), TM5;216-238(P), TM6;249-271(P), TM7;279-301(S)]

odorant receptor S1 [Mus musculus ] (AF121972);278/317 (87%)

Seq. ID. No. 610 [TM1;29-50(P), TM2;59-81(P), TM3;96-118(S), TM4;144-166(P), TM5;203-224(P), TM6;240-262(P), TM7;269-291(S)]

odorant receptor MOR83 [Mus musculus ] (AB030894);167/308 (54%)

Seq. ID. No.612 [TM1;56-78(S), TM2;97-119(S), TM3;136-158(P), TM4;184-206(P), TM5;239-261(P), TM6;278-299(S), TM7;310-331(S)]

odorant receptor S1 [Mus musculus ] (AF121972);195/293 (66%)

Seq. ID. No. 614 [TM1;26-48(P), TM2;60-82(S), TM3;93-115(S), TM4;141-163(P), TM5;206-228(P), TM6;238-259(S), TM7;268-290(S)]

olfactory receptor C6 [Mus musculus] (AF102523);148/299 (49%)

Seq. ID. No. 616 [TM1;19-41(S), TM2;62-84(S), TM3;100-122(S), TM4;140-162(P), TM5;202-224(P), TM6;242-264(S)]

BC62940_{—}2 [Homo sapiens] (AC004659);214/310 (69%)

Seq. ID. No. 618 [TM1;33-55(P), TM2;64-86(S), TM3;100-122(S), TM4;137-159(P), TM5;207-229(P), TM6;245-267(P), TM7;273-295(S)]

taste bud receptor protein TB 641 [Rattus norvegicus] (U50949);270/310 (87%)

Seq. ID. No. 620 [TM1;3-25(P), TM2;30-51(S), TM3;53-74(S), TM4;81-95(S),TM5;101-122(S),TM6;144-166(P),TM7;183-205(P), TM8;214-235(S)]

olfactory receptor [Callithrix jacchus] (AF127882);200/216 (92%)

Seq. ID. No. 622 [TM1;3-25(P), TM2;30-51(P), TM3;54-75(S), TM4;78-96(S),TM5;102-123(S),TM6;144-166(P),TM7;183-205(P), TM8;214-235(S)]

olfactory receptor [Eulemur rubriventer] (AF127861);200/216 (92%)

Seq. ID. No. 624 [TM1;16-38(P), TM2;94-116(S), TM3;140-162(P), TM4;171-193(S), TM5;200-222(P), TM6;239-260(P)]

similar to mouse olfactory receptor 13; similar to P34984 (PID:g464305) [Homo sapiens] (AC005587);217/307 (70%)

Seq. ID. No. 626 [TM1;1-23(P), TM2;52-74(P), TM3;120-142(S), TM4;168-190(P), TM5;230-251(P), TM6;262-284(P)]

odorant receptor MOR18 [Mus musculus ] (AB030895);158/299 (52%)

Seq. ID. No. 628 [TM1;27-49(P), TM2;62-84(S), TM3;98-120(S), TM4;145-167(P), TM5;205-226(P), TM6;237-259(P)]

odorant receptor MOR18 [Mus musculus ] (AB030895);149/293 (50%)

Seq. ID. No. 630 [TM1;39-61(P), TM2;80-102(S), TM3;115-137(P), TM4;160-182(P), TM5;190-212(S), TM6;225-246(P), TM7;261-283(P), TM8;288-310(P)]

similar to rat olfactory receptor OR18; similar to S29710 (PID:g423702) [Homo sapiens] (AC004908);164/302 (54%)

Seq. ID. No. 632 [TM1;31-53(P), TM2;62-84(S), TM3;101-123(S), TM4;133-155(S), TM5;164-186(S), TM6;200-222(P), TM7;238-260(P), TM8;268-290(S)]

odorant receptor MOR83 [Mus musculus ] (AB030894);164/307 (53%)

Seq. ID. No. 634 [TM1;27-49(P), TM2;92-114(P), TM3;133-155(P), TM4;200-222(P), TM5;237-258(S), TM6;266-288(S)]

odorant receptor MOR83 [Mus musculus ] (AB030894);262/306 (85%)

Seq. ID. No. 636 [TM1;5-27(P), TM2;33-55(S), TM3;59-81(S), TM4;83-105(S), TM5;114-136(S), TM6;144-166(P), TM7;180-201(S), TM8;214-236(S)]

taste bud receptor protein TB 641 [Rattus norvegicus] (U50949);189/244 (77%)

Seq. ID. No. 638 [TM1;7-29(S), TM2;67-89(S), TM3;102-124(P), TM4;166-188(S), TM5;209-231(P), TM6;237-259(S)]

BC319430_{—}5 [Homo sapiens] (AC006271);183/260 (70%)

Seq. ID. No. 640 [TM1;20-42(S), TM2;46-67(S), TM3;72-94(S), TM4;115-137(S), TM5;160-182(P), TM6;218-240(P), TM7;256-278(S), TM8;292-314(S)]

odorant receptor S1 [Mus musculus ] (AF121972);191/314 (60%)

Seq. ID. No. 642 [TM1; 2-24(S), TM2;48-70(P), TM3;116-138(S), TM4;162-184(P), TM5;188-210(S), TM6;222-244(S), TM7;263-285(S), TM8;325-345(P)]

OLF4 [Homo sapiens] (AC002988);200/302 (66%)

Seq. ID. No. 644 [TM1;25-47(P), TM2;58-80(S), TM3;100-122(P), TM4;142-164(P), TM5;193-215(P), TM6;241-262(S), TM7;272-293(S)]

BC85395_{—}3 [Homo sapiens] (AC005255);201/302 (66%)

Seq. ID. No. 646 [TM1;19-41(S), TM2;95-117(P), TM3;143-165(P), TM4;170-192(S), TM5;200-222(S), TM6;241-263(S)]

BC85395_{—}3 [Homo sapiens] (AC005255);186/298 (62%)

Seq. ID. No. 648 [TM1;27-49(P), TM2;60-82(S), TM3;100-122(P), TM4;143-165(P), TM5;210-232(P), TM6;243-265(S)]

olfactory receptor [Papio hamadryas] (AF127819);204/216 (94%)

Seq. ID. No. 650 [TM1;34-56(P), TM2;62-84(S), TM3;98-120(P), TM4;124-146(S), TM5;202-224(P), TM6;237-259(S), TM7;270-292(S)]

olfactory protein [Rattus norvegicus] (M64386);177/308 (57%)

Seq. ID. No. 652 [TM1;41-63(S), TM2;82-104(S), TM3;113-134(S), TM4;144-166(P), TM5;181-203(P), TM6;210-232(S)]

HGMP07J [Homo sapiens]>gi|2284811prf∥1804351C olfactory receptor HGMP07J [Homo sapiens] (X64995);124/248 (50%)

Seq. ID. No. 654 [TM1;12-34(P), TM2;45-67(S), TM3;101-123(S), TM4;144-166(S), TM5;197-219(P), TM6;240-261(S), TM7;272-293(S)]

HGMP07J [Homo sapiens]>gi|228481|prf∥1804351C olfactory receptor HGMP07J [Homo sapiens] (X64995);165/308 (53%)

Seq. ID. No. 656 [TM1;28-50(P), TM2;94-116(S),TM3;139-161(S), TM4;203-225(P), TM5;239-260(P), TM6;271-293(S)]

olfactory receptor P2 [Mus musculus ] (AF247657);155/308 (50%)

Seq. ID. No. 658 [TM1;51-73(P), TM2;83-105(P), TM3;156-178(P), TM4;202-224(P), TM5;229-250(P), TM6;265-287(P), TM7;292-314(P)]

odorant receptor MOR83 [Mus musculus ] (AB030894);174/304 (57%)

Seq. ID. No. 660 [TM1;1-23(S), TM2;50-72(P), TM3;81-103(S), TM4;113-135(P), TM5;160-182(S), TM6;222-244(P), TM7;258-280(S), TM8;292-314(S)]

olfactory receptor [Gorilla gorilla] (AF101764);135/306 (44%)

Seq. ID. No. 662 [TM1;23-45(P), TM2;60-82(S), TM3;98-120(S), TM4;139-161(P), TM5;203-225(P), TM6;237-259(S), TM7;271-292(S)]

olfactory receptor, family 12, subfamily D, member 2(NP_{—}039224);205/306 (66%)

Seq. ID. No. 664 [TM1;7-29(S), TM2;33-55(S), TM3;95-117(P), TM4;141-163(S), TM5;204-226(P), TM6;240-262(P), TM7;272-293(S)]

olfactory receptor [Mus musculus ] (AJ251155);157/309 (50%)

Seq. ID. No. 666 [TM1;18-40(P), TM2;55-77(S), TM3;111-133(P), TM4;142-164(P), TM5;195-217(P), TM6;246-268(P), TM7;284-305(S)]

candidate taste receptor T2R7(AF227133);95/303 (31%)

Seq. ID. No. 668 [TM1;8-30(P), TM2;45-67(S), TM3;97-119(S), TM4;128-150(P), TM5;180-202(P), TM6;228-250(P), TM7;252-273(S), TM8;276-298(S)]

candidate taste receptor T2R13(AF227137);140/307 (45%)

Seq. ID. No. 670 [TM1;7-29(P), TM2;48-70(S), TM3;99-120(S), TM4;129-151(P), TM5;178-200(P), TM6;227-249(P)]

candidate taste receptor T2R13(AF227137);136/306 (44%)

Seq. ID. No. 672 [TM1;10-32(P), TM2;42-64(P), TM3;93-115(S), TM4;126-148(P), TM5;182-204(P), TM6;235-257(P)]

candidate taste receptor T2R7(AF227133);131/311 (42%)

Seq. ID. No. 674 [TM1;19-41(S), TM2;61-83(S), TM3;108-130(S), TM4;138-160(P), TM5;195-217(P), TM6;247-269(P)]

candidate taste receptor T2R9(AF227135);101/307 (32%)

Seq. ID. No. 676 [TM1;34-56(P), TM2;75-97(S), TM3;114-136(P), TM4;158-180(P), TM5;209-231(P), TM6;262-284(P), TM7;286-308(S)]

taste receptor rT2R6(AF240766);100/291 (34%)

Seq. ID. No. 678 [TM1;34-56(P), TM2;75-97(S), TM3;114-136(P), TM4;158-180(P), TM5;209-231(P), TM6;258-280(P)]

candidate taste receptor T2R7(AF227133);103/310 (33%)

Seq. ID. No. 680 [TM1;7-29(P), TM2;94-116(P), TM3;131-153(P), TM4;182-204(P), TM5;235-257(P), TM6;267-289(P), TM7;291-307(S)]

taste receptor rT2R12(AF240768);214/307 (69%)

Seq. ID. No. 682 [TM1;8-30(P), TM2;46-68(P), TM3;91-113(S), TM4;128-150(P), TM5;180-202(P), TM6;229-251(P)]

candidate taste receptor T2R13(AF227137);140/309 (45%)

Seq. ID. No. 684 [TM1;8-30(P), TM2;45-67(P), TM3;90-112(P), TM4;129-151(P), TM5;178-200(P), TM6;227-249(P)]

candidate taste receptor T2R13(AF227137);116/246 (47%)

Seq. ID. No. 686 [TM1;7-29(P), TM2;34-56(P), TM3;61-83(P), TM4;116-138(P), TM5;147-169(P), TM6;197-219(P), TM7;248-269(P), TM8;295-317(S)]

candidate taste receptor T2R13(AF227137);131/292 (44%)

Seq. ID. No. 688 [TM1;8-30(P), TM2;45-67(P), TM3;97-119(P), TM4;126-148(P), TM5;178-200(P), TM6;230-251(P), TM7;267-289(S)]

candidate taste receptor T2R13(AF227137);136/309 (44%)

Seq. ID. No. 690 [TM1;8-30(P), TM2;45-67(P), TM3;97-119(P), TM4;128-150(P), TM5;180-202(P), TM6;229-251(P)]

candidate taste receptor T2R13(AF227137);115/252 (45%)

Seq. ID. No. 692 [TM1;8-30(P), TM2;46-68(S), TM3;99-121(S), TM4;128-150(P), TM5;184-206(P), TM6;229-251(P)]

candidate taste receptor T2R13(AF227137);136/306 (44%)

Seq. ID. No. 694 [TM1;8-30(P), TM2;45-67(P), TM3;97-119(S), TM4;128-150(P), TM5;178-200(P), TM6;228-250(P), TM7;276-298(S)]

candidate taste receptor T2R13(AF227137);131/293 (44%)

### INDUSTRIAL APPLICABILITY

The present invention makes it possible to search for novel GPCR genes or GPCR proteins which could discover a biocommunication system or identify a novel target protein for medicine cyclopaedically on a database. Further, by using the obtained GPCR proteins, it becomes possible to screen the endogenous ligands and the like. Said GPCR and their endogenous ligands are expected to be applicable to the research of medicines acting on them, and to the novel therapies, such as the application to gene therapies using said gene and its mutant. In addition, as the ligands of taste receptors and odorant receptors, they are expected to be applicable to develop a novel taste material, a bitter taste inhibitor, a novel odorant material, an odorant inhibitor and the like. Moreover, analysis of novel GPCR genes is expected to lead to discover the new biocommunication system as well as to identify novel target proteins for medicine.

1. A searching method of a G protein-coupled receptor gene and/or a G protein-coupled receptor protein characterized in extracting an open reading frame comprising 200 to 1500 amino acid residues and having 6 to 8 transmembrane segments from the genome data derived from a human, and in searching for a gene homologous to a known G protein-coupled receptor gene from the obtained open reading frame.

2. The searching method of a G protein-coupled receptor gene and/or a G protein-coupled receptor protein according to claim 1, wherein an open reading frame derived from a repeated sequence of DNA, an open reading frame with substantial undetermined amino acids, and an open reading frame in which any single amino acid residue comprising more than 20% of the whole sequence are eliminated, when extracting the open reading frame.

3. The searching method of a G protein-coupled receptor gene and/or a G protein-coupled receptor protein according to claim 1 or 2, wherein a gene homologous to a known G protein-coupled receptor gene is a G protein-coupled receptor gene or a G protein-coupled receptor associated gene.

4. The searching method of a G protein-coupled receptor gene and/or a G protein-coupled receptor protein according to any one of claims 1 to 3, wherein a G protein-coupled receptor contains an endogenous ligand.

5. The searching method of a G protein-coupled receptor gene and/or a G protein-coupled receptor protein according to claim 4, wherein a G protein-coupled receptor containing an endogenous ligand is a G protein-coupled receptor other than an odorant receptor or a taste receptor.

6. The searching method of a G protein-coupled receptor gene and/or a G protein-coupled receptor protein according to claim 4, wherein a G protein-coupled receptor containing an endogenous ligand is a G protein-coupled receptor of an odorant receptor.

7. The searching method of a G protein-coupled receptor gene and/or a G protein-coupled receptor protein according to claim 4, wherein a G protein-coupled receptor containing an endogenous ligand is a G protein-coupled receptor of a taste receptor.

8. A G protein-coupled receptor gene obtainable by the searching method of a G protein-coupled receptor gene and/or a G protein-coupled receptor protein according to any one of claims 1 to 7.

9. A gene which encodes a G protein-coupled receptor protein (a) or (b) described below;
(a) a G protein-coupled receptor protein comprising an amino acid sequence represented by Seq. ID No.2n (n=any one of integral numbers 1 to 51),
(b) a G protein-coupled receptor protein comprising an amino acid sequence where one or a few amino acids are deficient, substituted or added in an amino acid sequence represented by Seq. ID No.2n (n=any one of integral numbers 1 to 51).

10. DNA which encodes a G protein-coupled receptor protein comprising DNA which contains a base sequence represented by Seq. ID No. 2n-1 (n=any one of integral numbers 1 to 51) or its complementary sequence and a part or the whole of these sequences.

11. DNA which hybridizes with DNA comprising the gene according to claim 10 under a stringent condition, and encodes a G protein-coupled receptor protein.

12. A gene which encodes a G protein-coupled receptor protein (a) or (b) described below;
(a) a G protein-coupled receptor protein comprising an amino acid sequence represented by Seq. ID No. 2n (n=any one of integral numbers 52 to 332),
(b) a G protein-coupled receptor protein comprising an amino acid sequence where one or a few amino acids are deficient, substituted or added in an amino acid sequence represented by Seq. ID No. 2n (n=any one of integral numbers 52 to 332).

13. DNA which encodes a G protein-coupled receptor protein comprising DNA which contains a base sequence represented by Seq. ID No. 2n-1 (n=any one of integral numbers 52 to 332) or its complementary sequence and a part or the whole of these sequences.

14. DNA which hybridizes with DNA comprising the gene according to claim 13 under a stringent condition, and encodes a G protein-coupled receptor protein.

15. A gene which encodes a G protein-coupled receptor protein (a) or (b) described below;
(a) a G protein-coupled receptor protein comprising an amino acid sequence represented by Seq. ID No. 2n (n=any one of integral numbers 333 to 347),
(b) a G protein-coupled receptor protein comprising an amino acid sequence where one or a few amino acids are deficient, substituted or added in an amino acid sequence represented by Seq. ID No. 2n (n=any one of integral numbers 333 to 347).

16. DNA which encodes a G protein-coupled receptor protein comprising DNA which contains a base sequence represented by Seq. ID No. 2n-1 (n=any one of integral numbers 333 to 347) or its complementary sequence and a part or the whole of these sequences.

17. DNA which hybridizes with DNA comprising the gene according to claim 16 under a stringent condition, and encodes a G protein-coupled receptor protein.

18. A G protein-coupled receptor protein obtainable by the searching method of a G protein-coupled receptor gene and/or a G protein-coupled receptor protein according to any one of claims 1 to 7.

19. A G protein-coupled receptor protein comprising an amino acid sequence represented by Seq. ID No. 2n (n=any one of integral numbers 1 to 51).

20. A G protein-coupled receptor protein comprising an amino acid sequence where one or a few amino acids are deficient, substituted or added in an amino acid sequence represented by Seq. ID No. 2n (n=any one of integral numbers 1 to 51).

21. A G protein-coupled receptor protein comprising an amino acid sequence represented by Seq. ID No. 2n (n=any one of integral numbers 52 to 332).

22. A G protein-coupled receptor protein comprising an amino acid sequence where one or a few amino acids are deficient, substituted or added in an amino acid sequence represented by Seq. ID No. 2n (n=any one of integral numbers 52 to 332).

23. A G protein-coupled receptor protein comprising an amino acid sequence represented by Seq. ID No. 2n (n=any one of integral numbers 333 to 347).

24. A G protein-coupled receptor protein comprising an amino acid sequence where one or a few amino acids are deficient, substituted or added in an amino acid sequence represented by Seq. ID No. 2n (n=any one of integral numbers 333 to 347).

25. A partial peptide of a G protein-coupled receptor protein obtainable by the searching method of a G protein-coupled receptor gene and/or a G protein-coupled receptor protein according to any one of claims 1 to 7.

26. The partial peptide according to claim 25, wherein the G protein-coupled receptor protein is the G protein-coupled receptor protein according to any one of claims 19 to 22.

27. A fusion protein or a fusion peptide constructed by binding the G protein-coupled receptor protein according to claim 18 or the partial peptide of the G protein-coupled receptor protein according to claim 25 to a marker protein and/or a peptide tag.

28. The fusion protein according to claim 27, wherein the G protein-coupled receptor protein is the G protein-coupled receptor protein according to any one of claims 19 to 24.

29. An antibody which specifically binds to the G protein-coupled receptor protein according to claim 18.

30. The antibody according to claim 29, wherein the G protein-coupled receptor protein is the G protein-coupled receptor protein according to any one of claims 19 to 24.

31. A host cell containing an expression system which can express the G protein-coupled receptor protein according to claim 18.

32. The host cell according to claim 31, wherein the G protein-coupled receptor protein is the G protein-coupled receptor protein according to any one of claims 19 to 24.

33. A non-human animal wherein its function of a gene which encodes the G protein-coupled receptor protein according to claim 18 is deficient or said protein overexpresses on its chromosome.

34. The non-human animal according to claim 33, wherein the G protein-coupled receptor protein is the G protein-coupled receptor protein according to any one of claims 19 to 24.

35. The non-human animal according to claim 33 or 34, wherein the non-human animal is a mouse.

36. A screening method of a promoter or a suppressor of a G protein-coupled receptor function, or of a G protein-coupled receptor expression characterized in using the G protein-coupled receptor protein according to any one of claims 18 to 24, the partial peptide according to claim 25 or 26, or a cell membrane which expresses said protein or the partial peptide, and a test substance.

37. A screening method of a promoter or a suppressor of a G protein-coupled receptor function, or of a G protein-coupled receptor expression characterized in using the G protein-coupled receptor protein according to any one of claims 18 to 24, the partial peptide according to claim 25 or 26, or a cell membrane which expresses said protein or the partial peptide, a G protein or a partial peptide of a G protein, and a test substance.

38. A screening method of a promoter or a suppressor of a G protein-coupled receptor function, or of a G protein-coupled receptor expression characterized in using a cell expressing the G protein-coupled receptor protein according to any one of claims 18 to 24 or the partial peptide according to claim 25 or 26, and a test substance.

39. The screening method of a promoter or a suppressor of a G protein-coupled receptor function, or of a G protein-coupled receptor expression according to any one of claims 36 to 38, wherein a cell which expresses the G protein-coupled receptor protein according to any one of claims 18 to 24 or the partial peptide according to claim 25 or 26 is the host cell according to claim 31 or 32.

40. A screening method of a promoter or a suppressor of a G protein-coupled receptor function, or of a G protein-coupled receptor expression characterized in using the non-human animal according to any one of claims 33 to 35, and a test substance.

41. A promoter or a suppressor of a G protein-coupled receptor function, or of a G protein-coupled receptor expression obtainable by the screening method of a promoter or a suppressor of a G protein-coupled receptor function, or of a G protein-coupled receptor expression according to any one of claims 36 to 40.

42. The promoter or the suppressor of a G protein-coupled receptor function, or of a G protein-coupled receptor expression according to claim 41, wherein the promoter or the suppressor of a G protein-coupled receptor function, or of a G protein-coupled receptor expression is a ligand for a G protein-coupled receptor.

43. A medical constituent characterized in being used for a medical treatment for a patient who needs elevation of the function or enhancement of the expression of a G protein-coupled receptor, and containing the protein according to any one of claims 18 to 24, the partial peptide according to claim 25 or 26, or the promoter of a G protein-coupled receptor function or expression according to claim 41 or 42 as an active component.

44. A medical constituent characterized in being used for a medical treatment for a patient who needs suppression of the function or the expression of a G protein-coupled receptor, and containing the protein according to any one of claims 18 to 24, the partial peptide according to claim 25 or 26, or the suppressor of a G protein-coupled receptor function or expression according to claim 41 or 42 as an active component.

45. A diagnostic method for diseases relating to the function or the expression of a G protein-coupled receptor characterized in comparing a DNA sequence encoding a G protein-coupled receptor protein in a sample to a DNA sequence encoding the protein according to any one of claims 18 to 24.

46. A diagnostic probe for diseases relating to the function or the expression of a G protein-coupled receptor comprising the whole or a part of an anti sense strand of DNA or RNA encoding the protein according to any one of claims 18 to 24.

47. A diagnostic drug for diseases relating to the function or the expression of a G protein-coupled receptor characterized in containing the diagnostic probe according to claim 46 and/or the antibody according to claim 29 or 30.
